(19) United States
(12) Patent Application Publication
(10) Pub. No.: US 2019/0343686 A1
(43) Pub. Date: Nov. 14, 2019

(54) Devices for Application of Localized Hypothermic Therapy to the Human Ear

(71) Applicant: Restorear, LLC, Kirkland, WA (US)

(72) Inventor: Curtis S. King, Kirkland, WA (US)

(21) Appl. No.: 16/522,548

(22) Filed: Jul. 25, 2019

Related U.S. Application Data
(63) Continuation-in-part of application No. 16/163,479, filed on Oct. 17, 2018.
(60) Provisional application No. 62/573,716, filed on Oct. 18, 2017.

(51) Int. Cl. A61F 11/14; A61F 7/12; A61F 7/02; A61F 7/10; A61F 7/00

(52) U.S. Cl. CPC A61F 11/14; A61F 7/12; A61F 7/02; A61F 2007/108; A61F 7/007; A61F 2007/0005; A61F 2007/0075; A61F 7/10

(57) ABSTRACT

A heat transfer device particularly structured for application of thermal therapy from a contact surface to internal structures of a human ear. A device may be passive (pre-cooled), active (thermoelectrically active), or include elements of both. A device may be structured to apply thermal treatment from a contact surface of a contact cavity only to a localized posterior area relative to the circumference of an ear. A device may include both of a contact cavity and a bulk cavity, with heat transfer media disposed in each cavity. Typically, a bulk cavity holds at least twice the media volume contained in a contact cavity. The cavities may be disposed in fluid communication, or separated by a barrier to permit only thermal communication therebetween. When a barrier is present, a device may include different heat transfer media in each cavity. One or more device may be associated with a pair of auditory headphones to dispose a contact surface in contact with desired portion(s) of a head.

### RELATED APPLICATIONS

This application is a continuation-in-part of Utility Application Ser. No. 16/163,479, filed Oct. 17, 2018, for “Devices for Application of Localized Hypothermic Therapy to the Human Ear”, and claims the benefit under 35 U.S.C. 119(e) of the filing date of Provisional Application Ser. No. 62/573,716, filed Oct. 18, 2017, for “Devices and Methods for Application of Localized Hypothermic Therapy to the Human Ear”.

### BACKGROUND

### Field of the Invention

This invention relates to devices and methods for hypothermic therapy. It is particularly directed to devices and methods for application of hypothermic therapy to a human ear.

### State of the Art

It has long been known that application of hypothermic therapy (i.e., “therapeutic cooling”) of human body tissue can be beneficial in reducing inflammation and limiting adverse effects related to injury. Cooling therapy using e.g., ice, ice-packs, etc., has been used for centuries to treat injury. This trend continues today, and cooling therapy remains the first line of treatment for a variety of injuries. Consumer devices with different types of materials (gels, various polymeric fluids, etc.) are commercially available, and are commonly used. These devices are often designed with specific geometry or features such that the device, when installed on the human body, can efficiently cool the injured area, and reduce the severity of the inflammatory response.

In some instances, modern techniques utilizing therapeutic hypothermia have been developed, and have now become standard of care for advanced medical cases. It is commonly known that for patients suffering cardiac arrest, lowering the body temperature as quickly as possible after trauma offers significant benefits. In cases of traumatic central nervous system injuries (brain and spinal cord), localized hypothermic therapy (in these cases, referred to as Targeted Temperature Management) also shows promising results as a method for improved outcomes. Localized, or targeted application of, thermal therapy may avoid or reduce patient discomfort during the therapy.

Most recently, work in the field of otolaryngology and audiology has shown that there is a potential benefit to the use of this targeted/localized therapeutic hypothermia for the treatment of noise induced hearing loss, or hearing damage. Animal studies have shown that the application of localized cooling to the external regions of the skull nearest the ears (specifically, areas of the skull nearest the organs/structures of the outer, middle, and inner ear), following noise trauma may reduce or eliminate hearing damage. Data shows that application of this therapy may offer significant benefits for reducing hearing damage in both the long and the short term. Considering this, there exists a need for improved devices and methods for the effective application of therapeutic hypothermia to structures in, on, or around the region of a human ear.

### BRIEF SUMMARY OF THE INVENTION

Noise-induced hearing loss (NIHL) is an impairment resulting from irreversible damage to the hair cells or underlying neural structures in the cochlea as a result of noise exposure. The consequence of such damage is loss of hearing, that can occur acutely or over a period of time. The significant health problems presented by such hearing loss require development of new strategies to reduce or prevent it. We have shown that controlled and localized therapeutic hypothermia provided to the inner ear non-invasively and subcutaneously post-noise trauma conserves residual hearing. We have further shown that such a treatment may preserve sensitive neural structures against trauma. This is a novel technique that can be applied for preservation of hearing and balance during ototoxic insults, noise-exposure, traumatic brain injuries, exposures to blast, inner ear or middle ear surgeries, and chemotherapy. Cooling post-trauma can extend or postpone the critical time window of cell death by modulating multiple molecular and cellular pathways, which then allows for synergistic therapies. For example, one can envision local cooling after trauma to the ear (noise) “buying” the patient 24-48 hours. During this time, treatment can be combined with targeted drugs that are synergistic and provide a long-term benefit.

Certain embodiments structured according to the invention can be essentially described as an ice-pack for the ear. A system for treatment of a patient typically requires one thermal element or device per ear. Sometimes, a thermal device may be characterized as “passive”. An exemplary passive device includes a compartment, or bladder, containing some sort of thermal “working fluid” or thermal mass. Sometimes, an embodiment may be made reference to as “active”. An active device includes electronic elements to aid in cooling a localized area of a patient. Cooling devices may include both passive and active elements in workable combination. A cooling device may be structured for placement of the entire device, or only a portion thereof, into a cooling device such as a freezer to prepare a pre-chilled element for application of thermal therapy onto a patient.

In one method of use, the device(s) is/are placed into a freezer until the working fluid/mass attains a temperature significantly lower than human body temperature. With the fluid/thermal mass at low temperature, the device is placed on the skull, in a region near, or around the ear. The therapy occurs as the device cools the skull (and the auditory structures within the skull) locally, over a time period and at a rate dependent on the liquid volume, or thermal mass contained within the device.

An exemplary embodiment structured according to certain principles of this invention includes a thermal therapy device structured to interface in installed registration with an ear of a human head, the device being structured to place a heat transfer contact element in contact with the head at a localized area. In some cases, the localized area extends only partially around a circumference of the ear. Preferred devices are structured to couple with auditory headphones, so that a user may enjoy music or sonic therapy during thermal therapy sessions.

A therapy device may be structured to maintain an open pathway from the local environment to the ear canal of the ear on which the device is installed. In some cases, the open pathway, from the local environment to the ear canal of the ear on which the device is installed, passes through the device.

Desirably, the therapy device is structured to maintain the heat contact element at a posterior position with respect to the ear canal of the ear on which the device is installed. For example, an ear opening may be structured to cause radial compression against an exterior surface of the ear, the opening being structured to receive an ear in penetration there-through during installation of the device on the head. An exemplary such ear opening is generally ovaloid to generate a torque against a top and a bottom of an ear to resist twisting of the device about an axis perpendicular to the head. Typically, the ear opening extends around an entire circumference of an installed ear. In certain cases, the ear opening may include an ear cone affixed to the therapy device at a large diameter open end of the cone to dispose a free-standing conic element that extends to a smaller diameter opening disposed at the opposite end of the ear cone. An internal conic surface of the ear cone may be structured to stretch and accommodate in compression against an exterior surface of an installed ear.

Sometimes, the thermal therapy device is passive. Other times, the device may include an electrically active thermal element.

An exemplary heat transfer contact element is embodied in a floor of a contact cavity. Desirably, the floor is transversely flexible and conformable to accommodate and conform under compression against the skin in the vicinity of the ear. A therapy device may also include a bulk cavity disposed in thermal communication with the contact cavity. In certain cases, the bulk cavity is further disposed in fluid communication with the contact cavity. Desirably, the bulk cavity is sized to hold between two-times and about twenty five-times the volume of a heat transfer media that is held in the contact cavity (or even more, possibly much more).

In certain embodiments, a boundary between the contact cavity and the bulk cavity is defined by a step-change in cross-section at the boundary, and heat transfer between the contact cavity and the bulk cavity occurs across a cross-section disposed at the step-change location. Typically, the bulk cavity is insulated to resist heat transfer into the bulk cavity from the local environment. The bulk cavity may also be insulated to resist heat transfer into the bulk cavity from the head and/or structured to avoid contact with the head, and/or to resist heat transfer into the bulk cavity from the ear.

### BRIEF DESCRIPTION OF THE DRAWINGS

In the drawings, which illustrate what are currently considered to be the best modes for carrying out the invention:

FIG. 1 is a perspective view looking at the front side, or alternatively the head-side, or contact portion, of a localized cooling pack assembly structured according to certain principles of the invention;

FIG. 2 is a front plan view of the device in FIG. 1

FIG. 3 is a side view in elevation of the device in FIG. 1;

FIG. 4 is a close-up view of the detail indicated by circle 4 in FIG. 3;

FIG. 5 is an exploded assembly view in perspective looking at the back side of the embodiment in FIG. 1;

FIG. 6 is the cross-section view indicated in FIG. 2 by section lines 6-6 and looking in the direction of the arrows;

FIG. 7 is an exploded assembly view, similar to that in FIG. 5, of an alternative embodiment;

FIG. 8 is a rear plan view of the device in FIG. 7, partially assembled;

FIG. 9 is the cross-section view indicated in FIG. 8 by section lines 9-9 and looking in the direction of the arrows;

FIG. 10 is a rear plan view of a rear cover or bulk reservoir element;

FIG. 11 is the cross-section view indicated in FIG. 10 by section lines 11-11 and looking in the direction of the arrows;

FIG. 12 is a close-up view of the detail indicated by circle 12 in FIG. 11;

FIG. 13 is a plan view of a heat-sink reservoir element;

FIG. 14 is the cross-section view indicated in FIG. 13 by section lines 14-14 and looking in the direction of the arrows;

FIG. 15 is the cross-section view indicated in FIG. 13 by section lines 15-15 and looking in the direction of the arrows;

FIG. 16 is a close-up view of the detail indicated by circle 16 in FIG. 15;

FIG. 17 is a plan view of an insulator element;

FIG. 18 is a side view of the insulator element in FIG. 17;

FIG. 19 is the cross-section view indicated in FIG. 17 by section lines 19-19 and looking in the direction of the arrows;

FIG. 20 is a close-up view of the detail indicated by circle 20 in FIG. 19;

FIG. 21 illustrates a device according to certain principles of the invention installed with reference to an ear on a human head;

FIG. 22 is a plan view of another embodiment of a localized cooling pack assembly structured according to certain principles of the invention;

FIG. 23 is a perspective view of the embodiment in FIG. 22;

FIG. 24 is an exploded assembly view of the embodiment in FIG. 22;

FIG. 25 is another perspective view of the embodiment in FIG. 22;

FIG. 26 is a close-up view of a portion of the embodiment in FIG. 22, partially disassembled;

FIG. 27 is an exploded assembly view of a subassembly of the embodiment in FIG. 22;

FIG. 28 is a cross-section view of the device in FIG. 23, with the top and bottom covers removed;

FIG. 29 is a close-up view of the detail indicated by circle 29 in FIG. 28;

FIG. 30 illustrates another device according to certain principles of the invention installed with reference to an ear on a human head;

FIG. 31 illustrates another device according to certain principles of the invention installed with reference to an ear on a human head;

FIG. 32 is a perspective view illustrating an embodiment structured similar to conventional earphones;

FIG. 33 is a rear view in elevation of the embodiment in FIG. 32;

FIG. 34 is an exploded assembly view of the embodiment in FIG. 32;

FIG. 35 is a perspective view at the inside, or head-contacting part, of a cooling pack illustrated in FIG. 32;

FIG. 36 is a perspective view, slightly from above, illustrating an embodiment adapted for association with a helmet or hat;

FIG. 37 is a perspective view, slightly from below, looking at the embodiment of FIG. 36;

FIG. 38 illustrates the embodiment of FIG. 36 installed on a human head;

FIG. 39 is a perspective view of a human head indicating a preferred area for application of therapeutic cooling;

FIG. 40 is a cross-section through a human head illustrating the internal structures effected by cooling therapy;

FIG. 41 is a plan view of an exemplary mobile device for use in combination with certain preferred embodiments;

FIG. 42 illustrates an exemplary and workable hardware/software architecture;

FIG. 43 illustrates communication between an exemplary mobile device and an exemplary therapeutic device;

FIG. 44 is an exploded assembly view of a cooling ear cup prior to its assembly onto a commercially available set of auditory headphones;

FIG. 45 is a perspective view of a set of auditory headphones with installed cooling ear cups;

FIG. 46 is a close-up view of one ear phone and an assembled cooling ear cup;

FIG. 47 is a perspective view of the cooling ear cup in FIG. 44;

FIG. 48 is a front (patient contact side) view of the cooling ear cup in FIG. 47;

FIG. 49 is a side view of the cooling ear cup in FIG. 47;

FIG. 50 is a cross-section view taken through section 50-50 in FIG. 51 and looking in the direction of the arrows;

FIG. 51 is a rear view of the cooling ear cup in FIG. 47;

FIG. 52 is an exploded assembly view of another cooling ear cup embodiment prior to its installation on an alternative commercially available set of auditory headphones;

FIG. 53 is a perspective view of the headphones in FIG. 52, with a pair of cooling ear cups installed;

FIG. 54 is the cross-section view indicated by section 54-54 in FIG. 53, and looking in the direction of the arrows;

FIG. 55 is a close-up view of the portion in FIG. 54 indicated by circle 55;

FIG. 56 is a perspective front view (patient contact side) of the cooling ear cup in FIG. 52;

FIG. 57 is a front view of the cooling ear cup in FIG. 56;

FIG. 58 is a side view of the cooling ear cup in FIG. 56;

FIG. 59 is a rear view of the cooling ear cup in FIG. 56;

FIG. 60 is the cross-section view indicated at section 60-60 in FIG. 59, and looking in the direction of the arrows; and

FIG. 61 is a close-up view of the portion indicated by circle 61 in FIG. 60.

### DETAILED DESCRIPTION OF THE ILLUSTRATED EMBODIMENTS

Reference will now be made to the drawings in which the various elements of the illustrated embodiments will be given numerical designations and in which the invention will be discussed so as to enable one skilled in the art to make and use the invention. It is to be understood that the following description is only exemplary of certain principles of the present invention, and should not be viewed as narrowing the claims which follow.

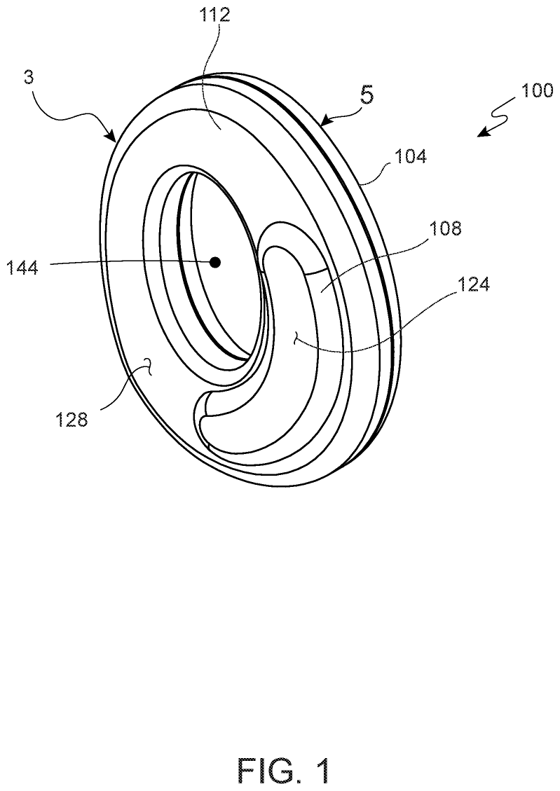

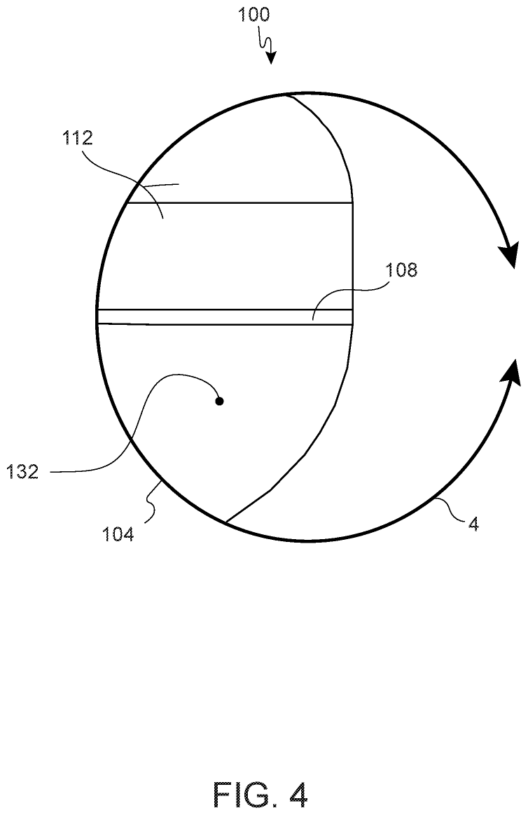

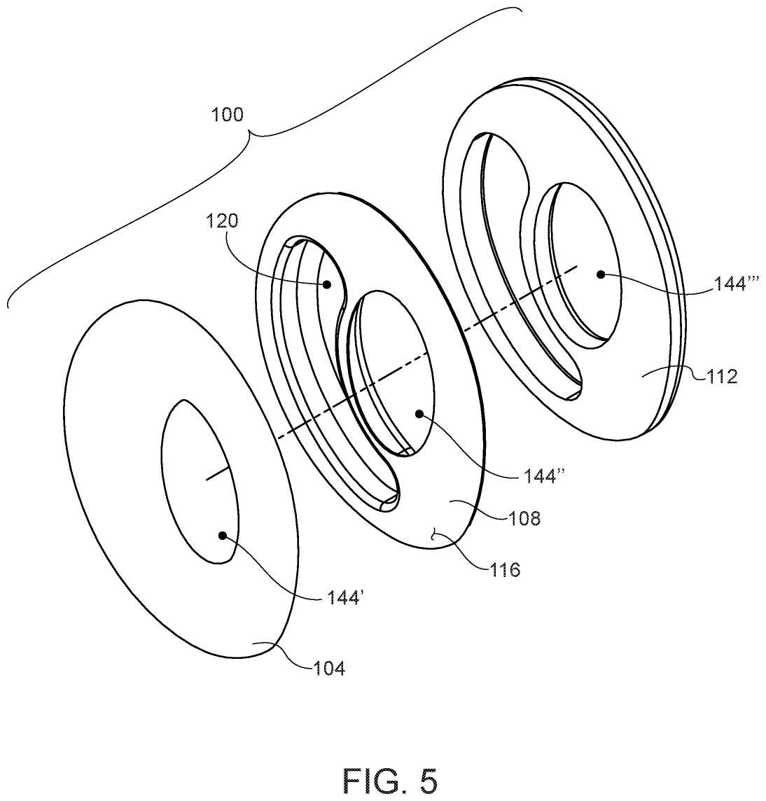

An exemplary thermal device, or cooling pack assembly, is indicated generally at 100 in FIGS. 1 through 6. With particular reference to FIG. 5, embodiment 100 includes a rear cover 104, a membrane 108, and an insulator 112. Membrane 108 includes a substantially flat surface 116 that is formed to include or define a cavity 120. Cover 104 forms a rear surface of device 100.

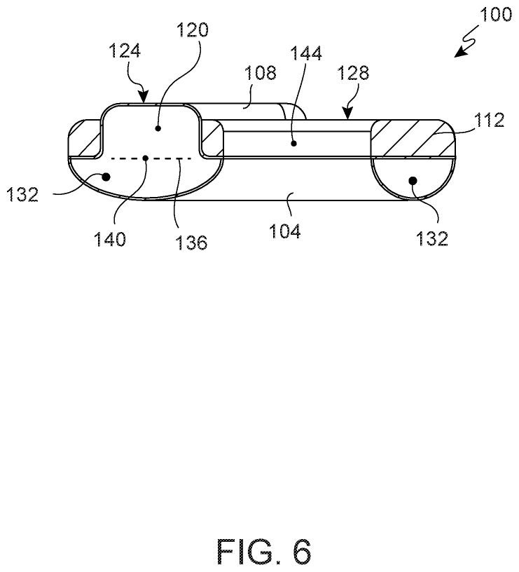

Cavity 120 forms a contact heat transfer reservoir, and typically projects through insulator 112 to dispose its front or contact surface 124 proud of the exposed front surface 128 of insulator 112 (see FIG. 6), such that surface 124 will contact the patient's skin near the ear. Desirably, cavity 120 provides a transversely flexible contact surface 124 that is structured to conform to the different anatomical surfaces of a variety of different patients. Also, it is desirable for membrane 108 to facilitate heat transfer from a patient into thermal/heat transfer fluid or media confined inside cavity 120.

As illustrated in FIG. 6, cover 104 forms a cavity 132 in which to hold a bulk quantity of heat transfer fluid or media. Desirably, cover 104 includes an insulating property to resist heat transfer from a patient's ear (e.g., from a medial portion of the projecting cantilevered ear “flap” or helix), or the local environment, into cavity 132. Consequently, a cover 104 may include a plurality of layers of different materials (not illustrated). Cavity 120 and cavity 132 may be regarded as separated by an imaginary boundary 136 for purpose of structure definition. In fact, and in the case of illustrated embodiment 100, they may be in fluid communication and together form a combined reservoir cavity 140, in which is confined heat transfer media or fluid. It is preferred for a cavity 132 to be sized to hold a volume of heat transfer media that is about 2 times as great as a volume held in cavity 120. A workable cavity 132 may hold between 2 times and up to perhaps 25 times the volume that is held in a cavity 120, or even more.

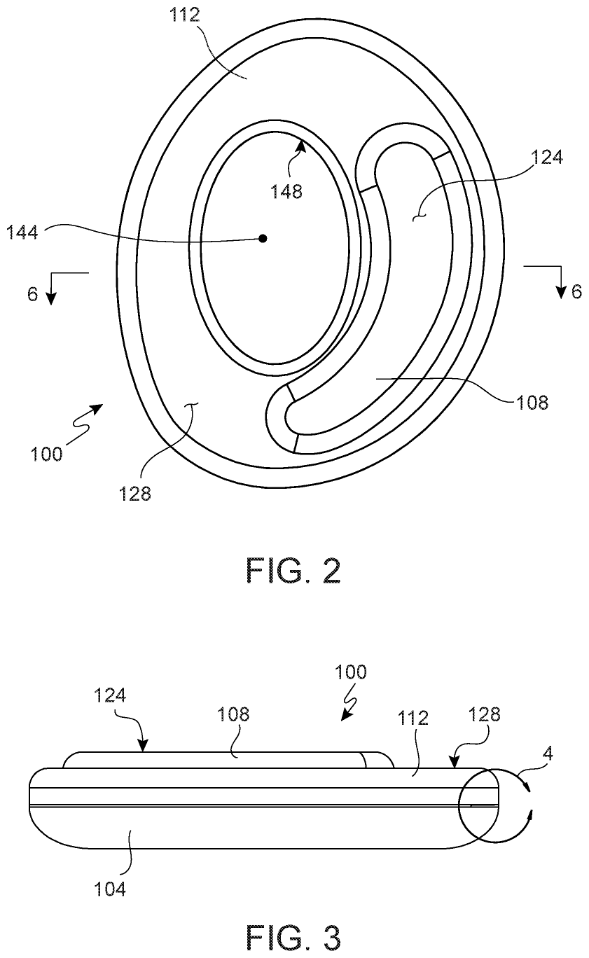

A through-hole 144 is formed in device 100 to accommodate passage there-through of a human ear. Through-hole 144 is formed by corresponding through-holes 144′, 144″, and 144′″ in cover 104, membrane 108, and insulator 112, respectively (see FIG. 5). As shown in FIG. 2, the perimeter 148 of through-hole 144 may be structured as a generally ovaloid cross-section, to fit in registration with the conventional shape of a human ear when the device 100 is installed on an ear. The non-circular cross-section of through-hole 144 resists twisting of the device out of desired registration to apply cooling to a particular location of the head.

Embodiment 100 is a passive cooling pack, and includes a cavity 140 that contains heat transfer media or fluid (liquid, gel, etc.). The entire device 100, or a component such as cavity 120, is typically chilled in a freezer, then applied as desired to a patient's head in the vicinity of an ear. Heat is removed from a localized portion of the patient's head, and absorbed by contact heat transfer reservoir 120. Heat gained in reservoir 120 is then transferred into bulk reservoir 132.

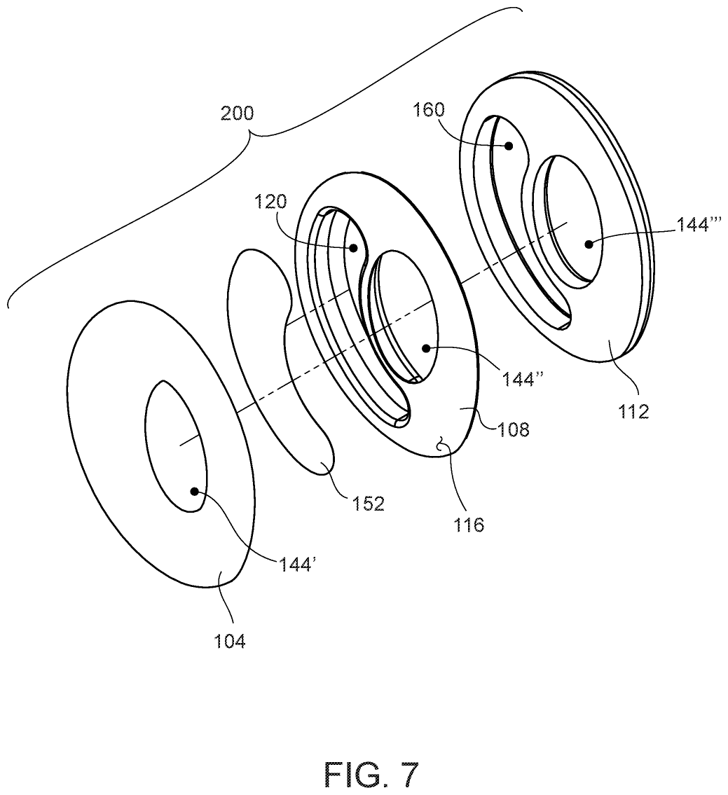

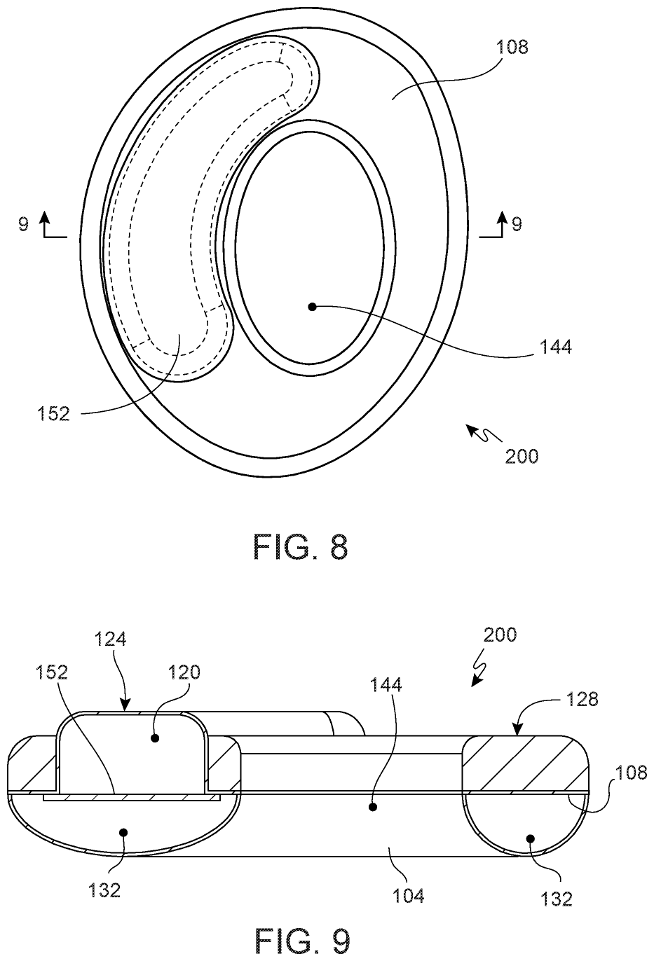

A two-reservoir embodiment is indicated generally at 200 in FIGS. 7-9. Embodiments similar to 100 and 200 may include certain elements in common, which are generally numbered accordingly. Superficially, embodiment 200 may appear to be identical to embodiment 100 in e.g., FIG. 1. However, embodiment 200 includes an internally-disposed physical barrier element 152 to define a fluid-tight separation between contact cavity 120 and bulk cavity 132. FIG. 8 illustrates barrier 152 stacked in registration on top of membrane element 108 to form a cap for cavity 120. Desirably, barrier element 152 facilitates heat transfer between media confined in respective cavities 120 and 132. Embodiment 200 therefore provides an opportunity to include different heat transfer media in each cavity. That permits tailoring a heat transfer profile or behavior for embodiment 200 compared to that available with embodiment 100.

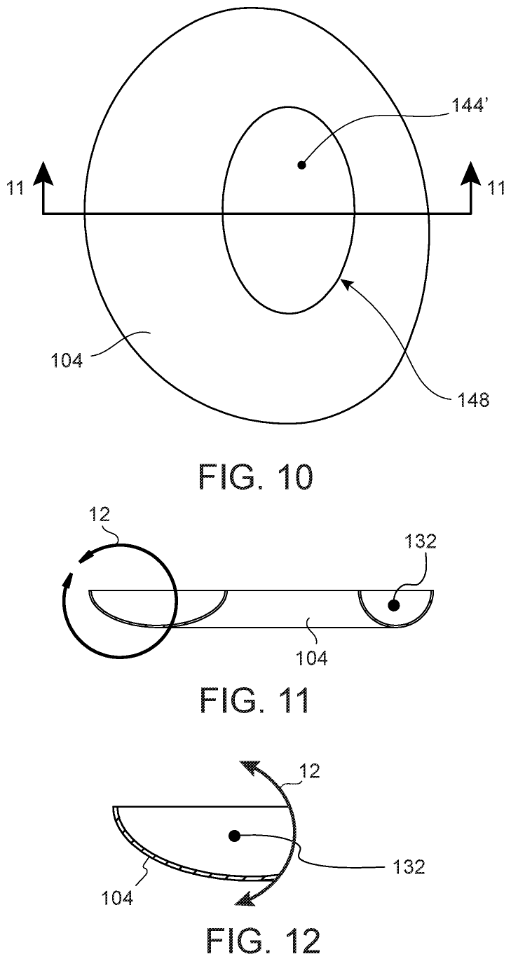

Certain details of a workable rear cover 104 are illustrated in FIG. 10-12. A workable cover 104 may be formed from a flexible membrane, such as a polymer film, or from a rigid plastic, hard or firm rubber, urethane, and the like. Certain covers 104 may expand like a balloon to accommodate loading or thermal expansion e.g., of heat transfer media. A workable rear cover 104 may sometimes be insulated to resist heat transfer from the environment into the confined heat transfer media. Certain rear covers may encompass a plurality of layers of different materials to provide a desired functionality, such as resistance to heat transfer or to physical damage.

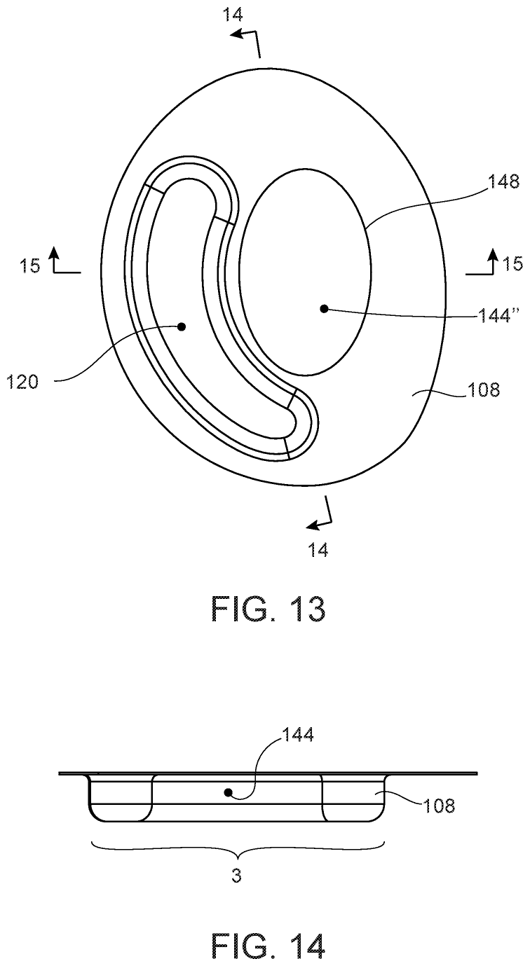

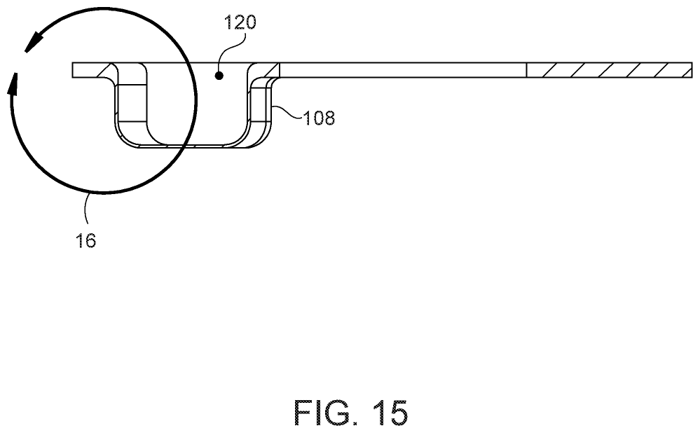

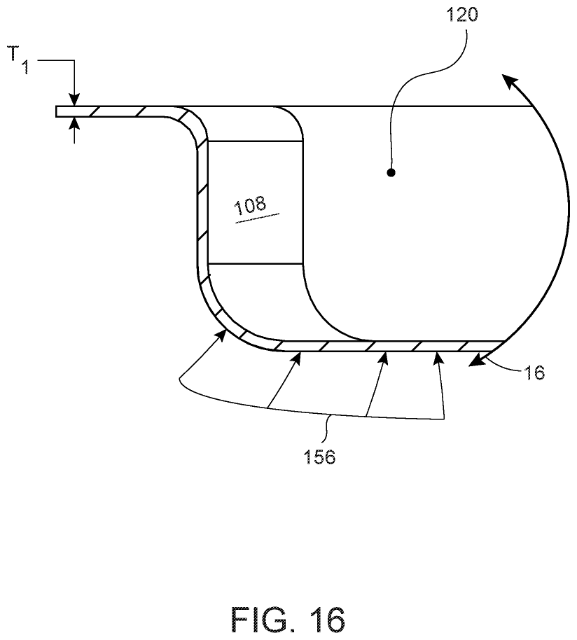

Certain details of a workable membrane 108 are illustrated in FIGS. 13-16. A membrane may be fashioned by heat-forming, blow-molding, injection molding, or other known manufacturing method to generate a cavity defined by a membrane or thin wall. A currently preferred membrane 108 is made from polymer film having a thickness T₁ (see FIG. 16) of about 0.005-0.010″ thickness. A preferred material includes a stretchable polymer film (e.g., polyethylene (LDPE, HDPE, LLDPE), polyester, nylon, Teflon, etc.). Such material may be used to manufacture either/both of cavities 120 and 132. The membrane/film desirably providing a transversely deformable wall to contact and conform to a patient's head responsive to an applied pressure profile 156. The membrane forming patient-contact cavity 120 also preferably facilitating heat transfer there-through.

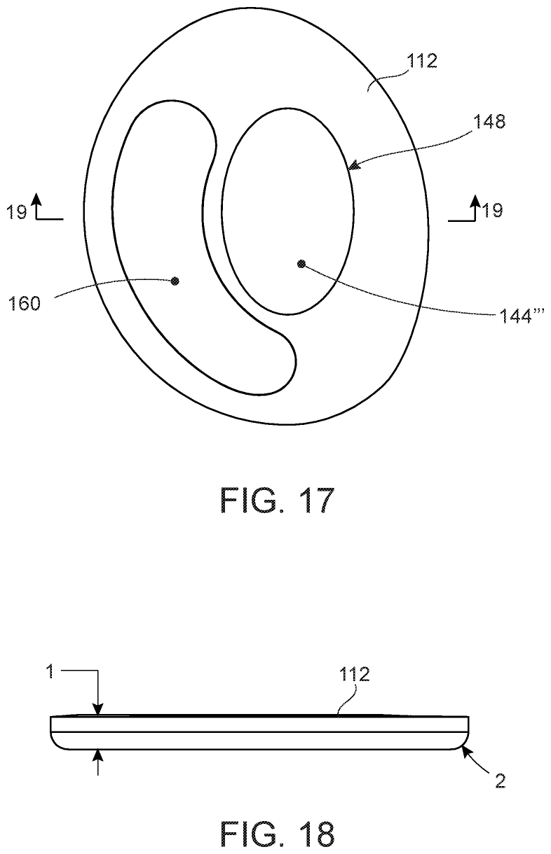

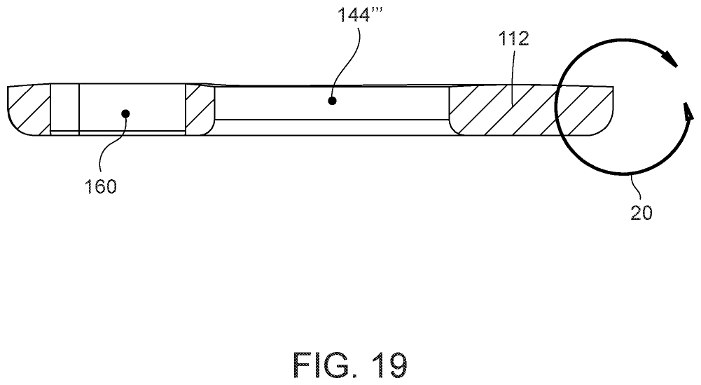

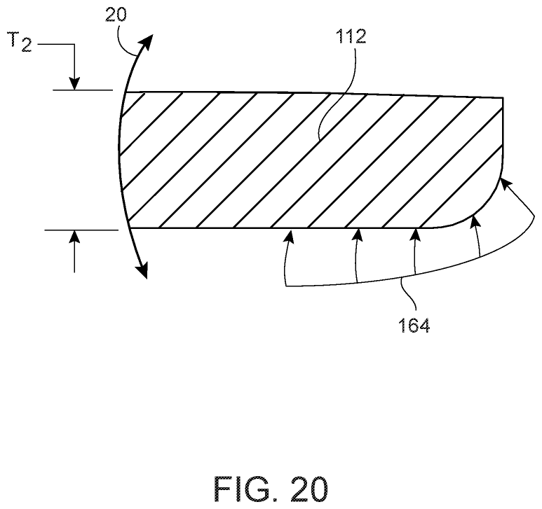

Certain details of a workable insulator 112 are illustrated in FIGS. 17-18. A workable insulator resists heat transfer, and may be manufactured from open-cell foam, thermally-resistant material, skinned poly foam, or the like. Portions of insulator 112 also contact a patient's head, and are typically deformable or may be pre-shaped in a variety of different assemblies to comfortably accommodate between head shapes of different patients. The insulator 112 desirably providing a deformable surface to contact and conform to a patient's head responsive to an applied pressure profile 164. A currently preferred insulator 112 is made from foam having a thickness T₂ (see FIG. 20) of about 0.125-0.5″ in thickness. A workable material to form an insulator 112 includes open/closed cell, thermally insulating polymer foam or rubber (closed cell/rubber, e.g., EPDM, neoprene, silicone, PVC, and polypropylene; open cell, e.g., polyurethane foam, open cell rubber).

The various elements may be fused together thermally, joined mechanically, or bonded with adhesives, and the like. Alternative manufacturing methods and suitable materials of construction will be apparent to one of ordinary skill in the art.

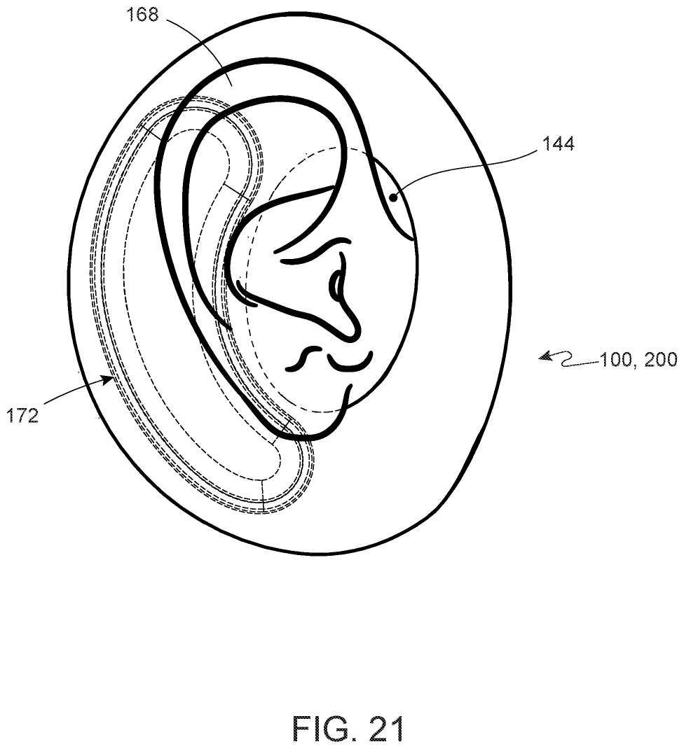

With reference now to FIG. 21, an embodiment such as 100 or 200 is illustrated in installed registration with respect to an ear 168 of a patient. Note that a portion of an installed device 100, 200 is desirably located in closest achievable proximity to the ear canal and between the ear “flap”, or helix, and the skin covering the patient's skull. One desired localized area of applied cooling (heat extraction or heat removal from the patient's head) is indicated by phantom-line structure indicated at 172.

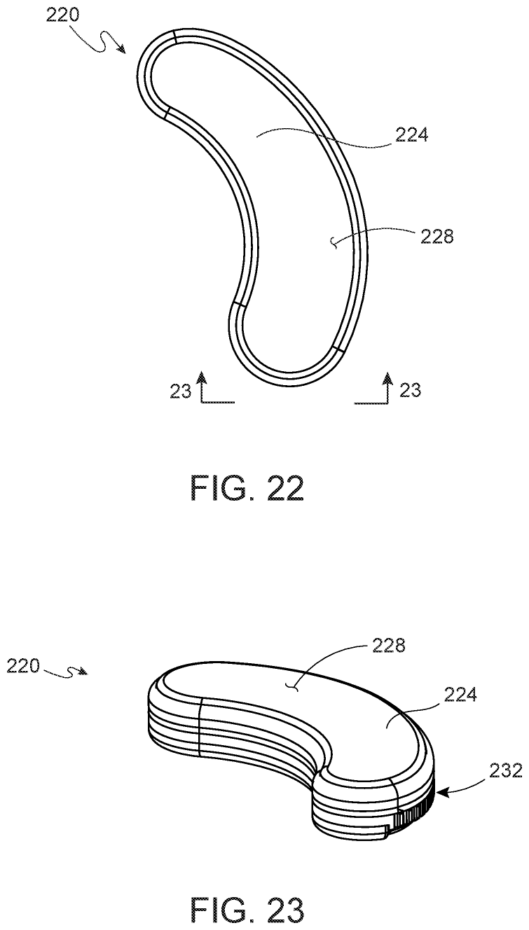

Another embodiment is indicated generally at 220 and is described with reference to FIGS. 22-29. Embodiment 220 is an active device, and includes electronic components that provide heat transfer from a localized area of a patient's head. An active device such as 220 may sometimes include one or more passive component, which can be pre-cooled (e.g., frozen). Certain active devices 220 may extend thermal therapy for a time beyond the capability of a passive device, and may provide a desired programmable and time-variable cooling profile to a patient.

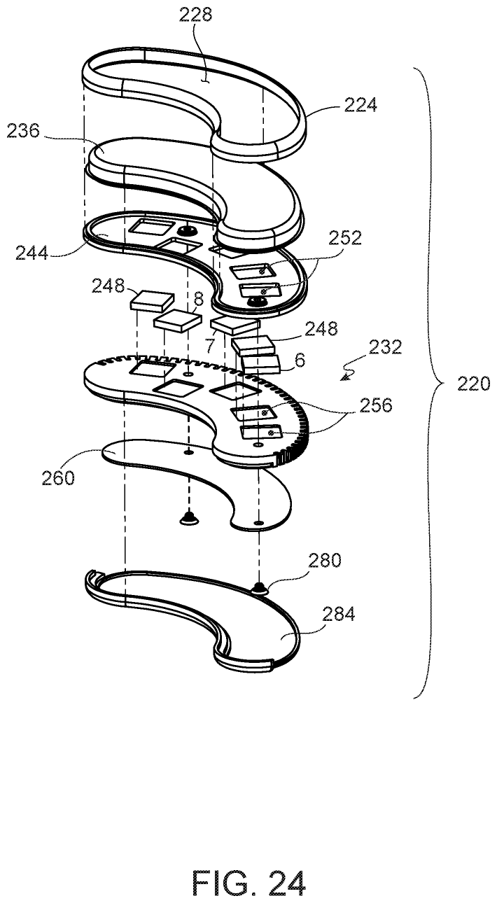

Active device 220 includes a front cover 224 with a surface 228 structured and disposed for contact to the skin surface behind a patient's ear. A preferred cover 224 may be characterized as including a membrane portion that is transversely flexible for accommodating to different head shapes of a plurality of patients. A heat-dissipating element, generally 232, is structured to dump heat from the device 220 to the local environment. An operable heat-dissipating element includes a series of fins and gaps provided in a thermally conductive heat sink material. With particular reference to FIGS. 24 and 27, a bladder 236 is installed in registration in contact with cover 224, and functions to draw heat from the patient's localized therapy area. A bladder 236 carries a heat transfer media, and may be pre-cooled (e.g., frozen like a passive ice pack). Bladder 236 is spaced apart from a heat sink element 240 by an insulating layer 244.

One or more (as illustrated, a plurality) thermoelectric heat transfer element 248 can be disposed to transport heat from the bladder 236 to the heat sink element 240. A workable heat sink element 240 may be manufactured from metal. A workable thermoelectric element 248 includes a Peltier device. A cooperating window 252 provides through-penetration of a device 248 and permits the cold side of an element 248 to contact and extract heat from the bladder 236. Sometimes, registration structure, such as a socket 256, may be provided as a manufacturing assembly aide to locate a thermoelectric device 248 with respect to the heat sink element 240. In general, a workable registration structure facilitates orientation of one component or element with respect to another component or element.

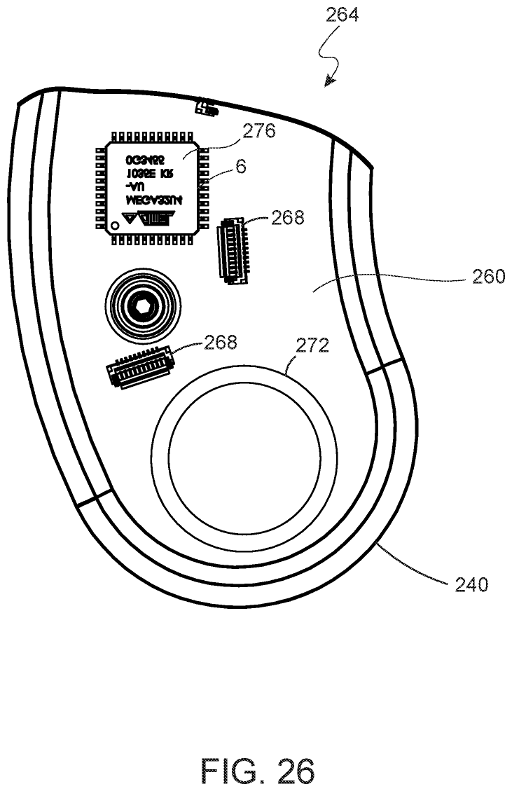

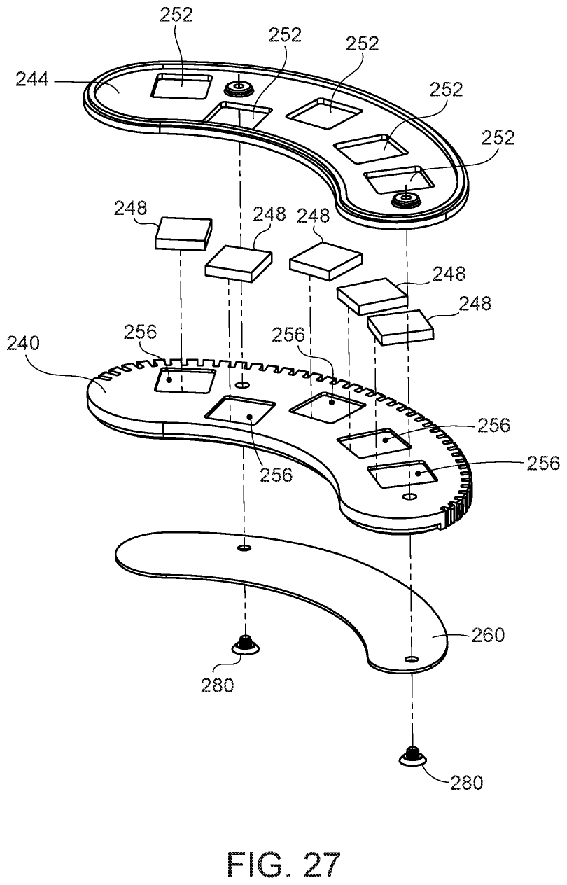

The thermoelectric element(s) 248 are disposed in electrical communication with circuit board 260, which carries the electrical components (generally indicated at 264 in FIG. 26) for operation and control of the device 220. Electrical components that may be carried by circuit board 260 include: electrical connectors 268 to communicate with elements 248; on-board power supply, such as battery 272; and a micro-controller or integrated circuitry 276. The assembly or subassembly may be held together, at least in part, by one or more fastener 280.

A rear cover 284 provides a protective closure for the device 220. Desirably, rear cover 284 provides an insulation and spacing function to resist contact by the patient with heat sink element 240. In the illustrated embodiment, rear cover 284 is bonded around a portion of its perimeter to front cover 224.

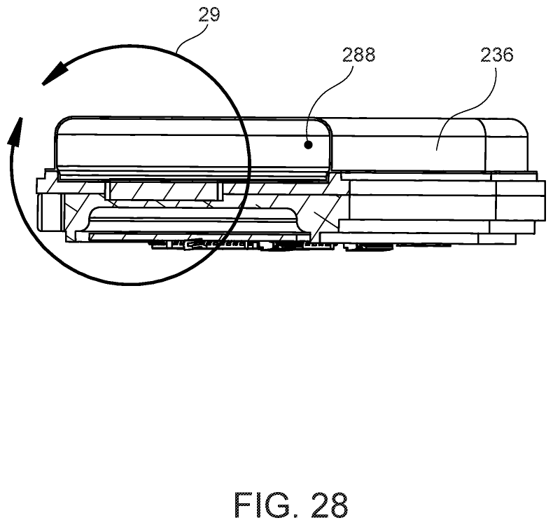

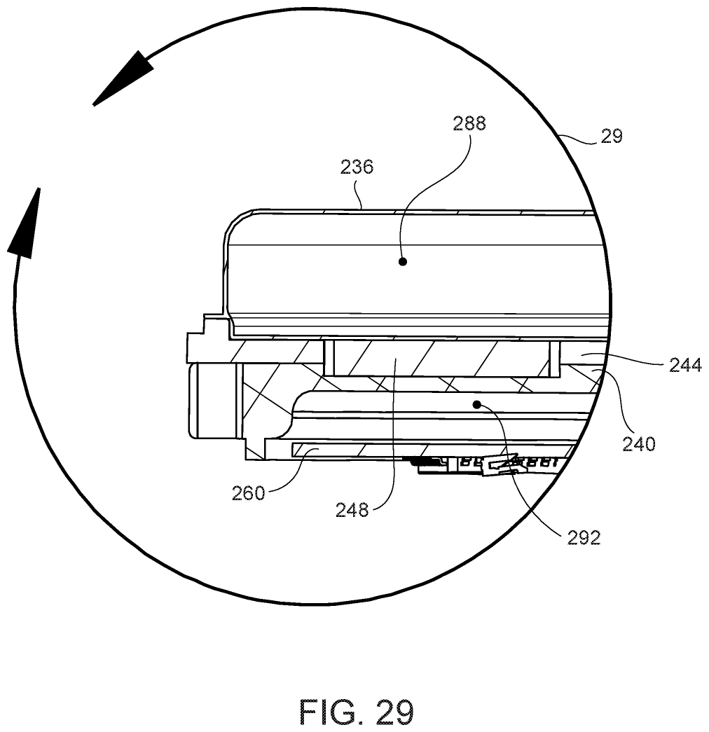

With reference now to FIGS. 28 and 29, it can be seen how the various electronic and mechanical elements cooperate to cool heat transfer media confined inside the volume 288 defined by a bladder 236. An air gap 292 is provided to facilitate performance of the heat dissipation structure 232. Currently, air circulation through gap 292 is promoted by convection. Incorporation of a fan to drive air flow is within contemplation.

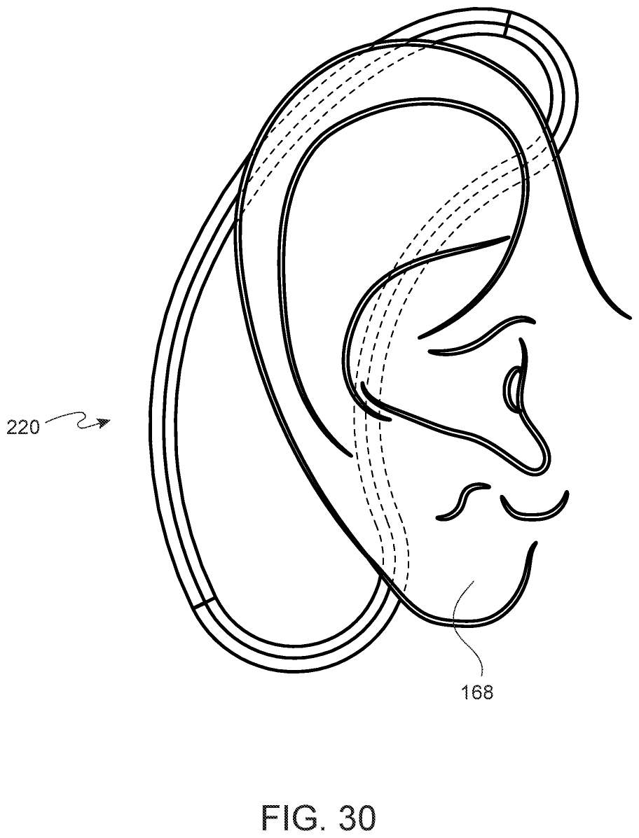

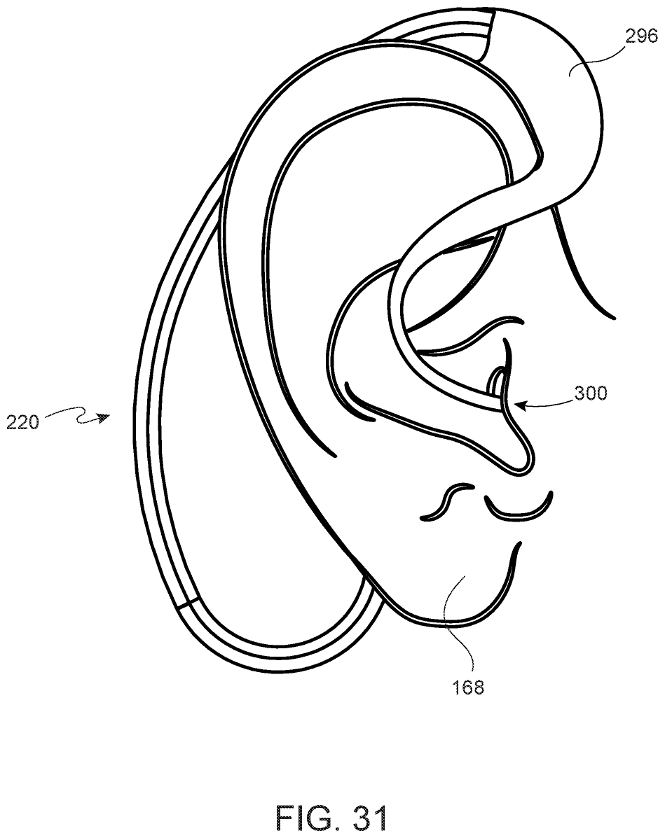

FIG. 30 illustrates an active embodiment 220 installed in registration with an ear 168 on a human head. An active embodiment 220 may alternatively be replaced by an entirely passive thermal therapy device having a cooperating size and configuration. An embodiment with an external configuration similar to that illustrated in FIG. 30 may be temporarily bonded onto the skin posterior to a patient's ear, or secured with tape or a wrap, or other workable retention structure. FIG. 31 illustrates optional mounting structure 296 affixed to a thermal therapy device and extending over the lateral side of the ear 168. In certain cases, and as illustrated, mounting device 296 may include a portion, generally 300, that extends into the ear canal for additional installation security. Mounting structure 296 may alternatively terminate with structure shaped to register with any suitable, cooperating, and laterally-exposed portions of structure of the patient's ear (loops, swirls, sockets, cavities, etc.).

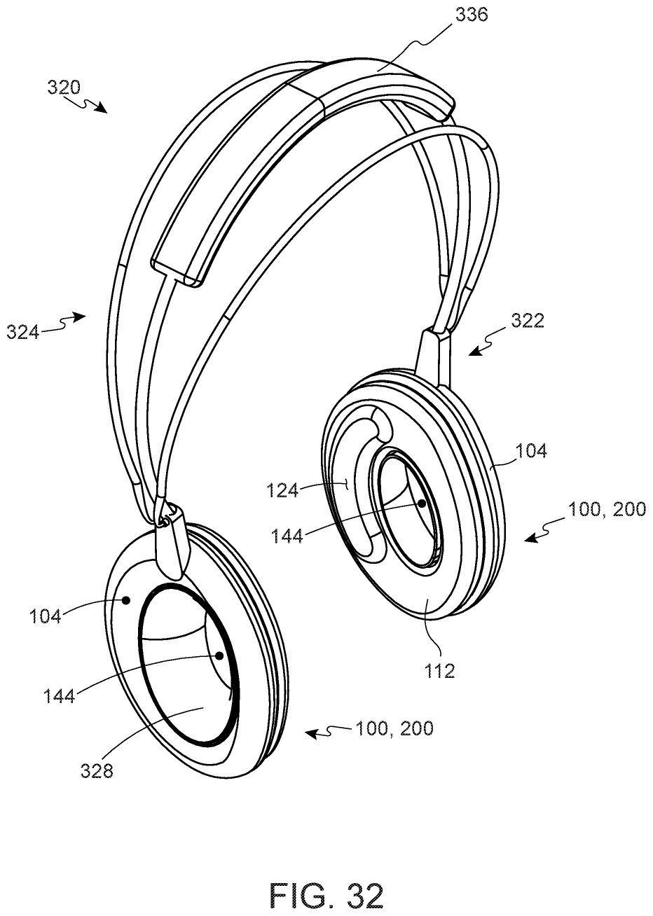

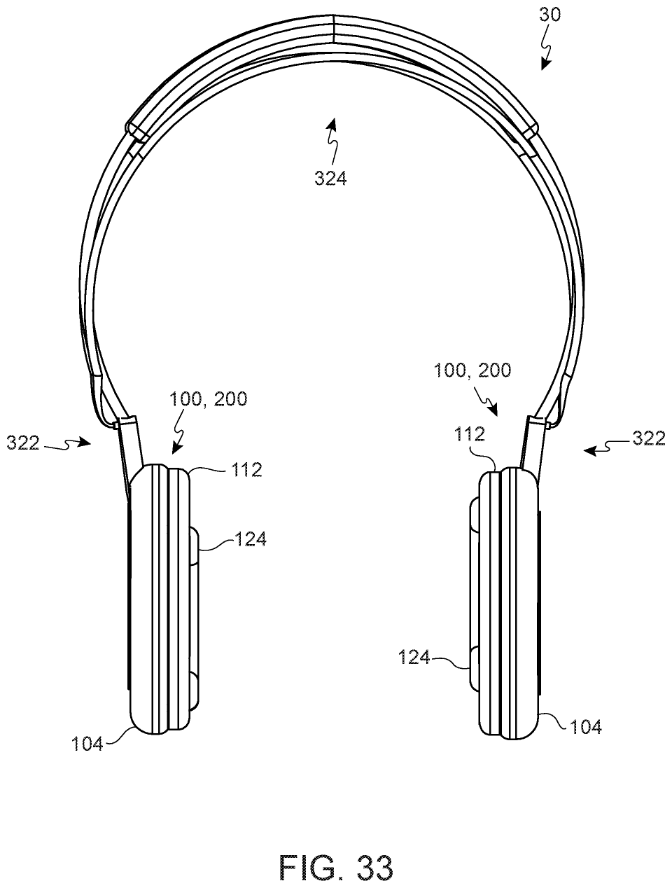

The embodiment indicated generally at 320 in FIGS. 32-35 includes a pair of thermal therapy packs associated with a head band 324, similar to conventional stereo headphones. Although the illustrated thermal packs are externally structured according to embodiments 100 and 200, it is within contemplation that one or both may be replaced with an embodiment including an active heat transfer element.

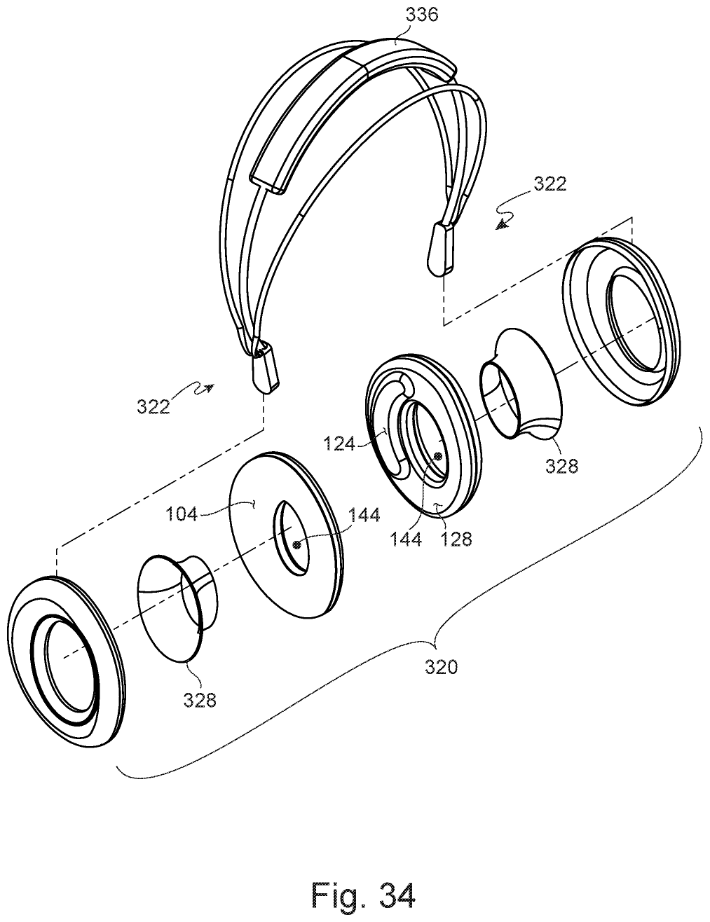

Of note, embodiment 320 also illustrates optional ear cone elements 328. Cone elements 328 are structured from resilient elastic material that may stretch to facilitate installation of an ear there-through, and to facilitate comfort of an installed device. Desirably, the cone elements 328 improve a grip on outer structure of an ear 168 by an installed thermal therapy device. During installation, an ear helix is drawn through the opening 144 of each cooling pack. It is currently desired for the ear cones 328 to be open at each end, to avoid interfering with the hearing of a patient that is undergoing thermal therapy. An operable ear cone 328 can be affixed to a thermal therapy device at a large diameter open end, leaving a free-standing conic section that extends to a smaller diameter opening disposed at the opposite end of the cone. The smaller diameter conic opening may facilitate positioning of a cooling portion 124 in registration near the base of the ear/skull junction, as well as to help grip the ear near its cantilever base. As illustrated in e.g., FIG. 34, an ear cone 328 may be affixed to a shell element 330, and the shell element 330 may be associated with a thermal therapy device. Other arrangements will be readily apparent to one of ordinary skill.

Connection structure, generally 332, may be included to facilitate coupling/decoupling a cooling pack to the headband 324. Connection structure 332 permits removal of a device for pre-cooling without exposing the entire assembly 320 to thermal change and potential temperature or condensate-induced damage. Desirably, a cooling device 100, 200, etc., is mounted with respect to the headband 324 to permit adjusting the device and band 324 to fit a device comfortably in registration on both sides of a patient's head. Typically, that includes degrees of freedom for rotation of each device about horizontal and vertical axes, and a length adjustment of band 324 between the devices. Also, headband 324 may include a compartment 336 in which to dispose electronics, power supply, wireless communication, control circuitry, and the like.

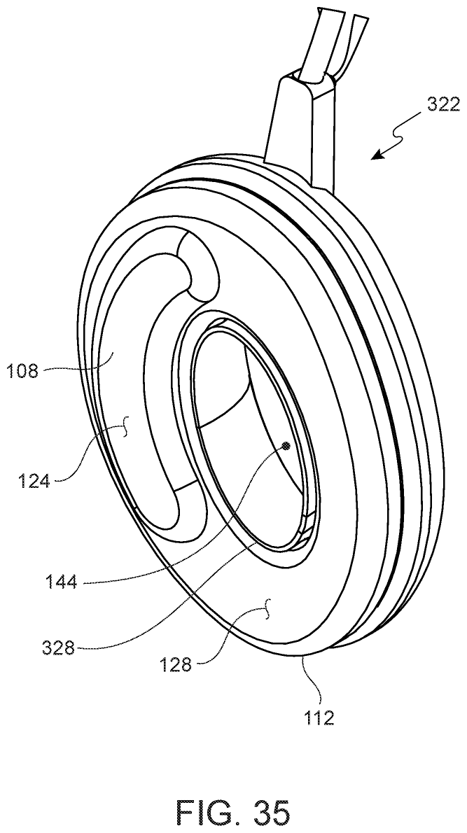

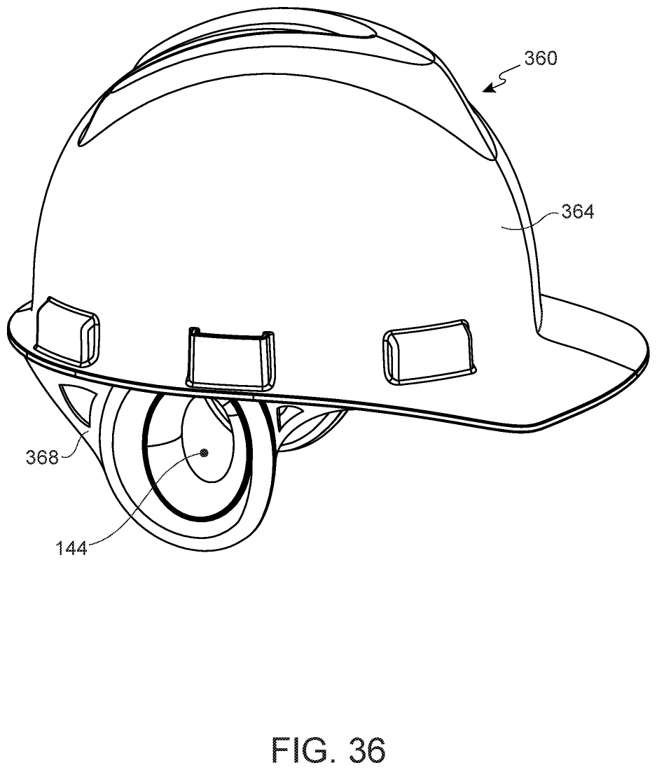

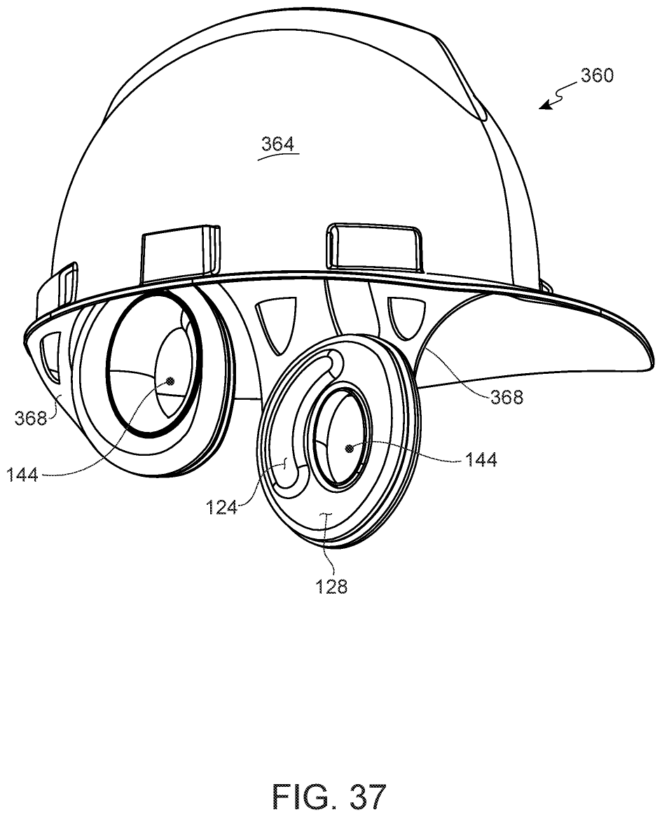

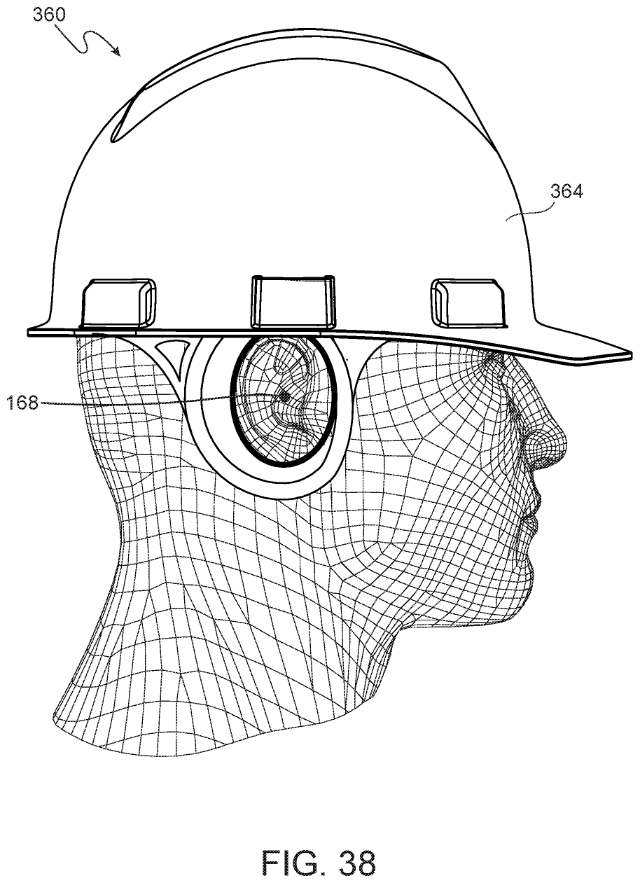

FIGS. 36-38 illustrate an alternative assembly, generally 360, including a helmet 364 structured to carry a pair of thermal therapy devices for installation on a human head. A cooling pack mounting band 368 may be adapted to removably hold a pack in position for desired therapy. Desirably, the pack is removable (and sometimes may even be partially dis-assembled) for safe cooling or freezing of one or more removed element.

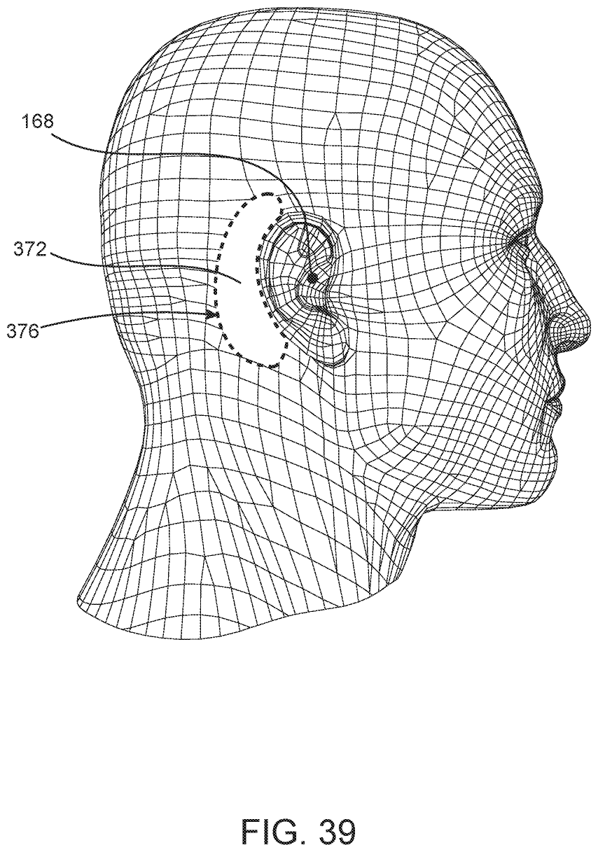

FIG. 39 illustrates a desired localized therapy area 372 that is bounded by a perimeter 376. Area 372 is roughly bean-shaped, and may be characterized as an arcuate shape having a center of curvature on the earhole side and extending partially around a circumference of an ear. Area 372 is desirably disposed to approximately abut a posterior ear surface, substantially as close to the ear canal as a thermal therapy device can comfortably fit. It is currently preferred to apply thermal therapy (cooling) only in a localized area, such as the localized area 372. Consequently, a contact heat transfer element e.g., cavity 120, is shaped to fit within the perimeter 376. Workable contact shapes for heat transfer patient contact surface 124 include round, rectangular, and arcuate to generally match the bean shape defined by perimeter 376.

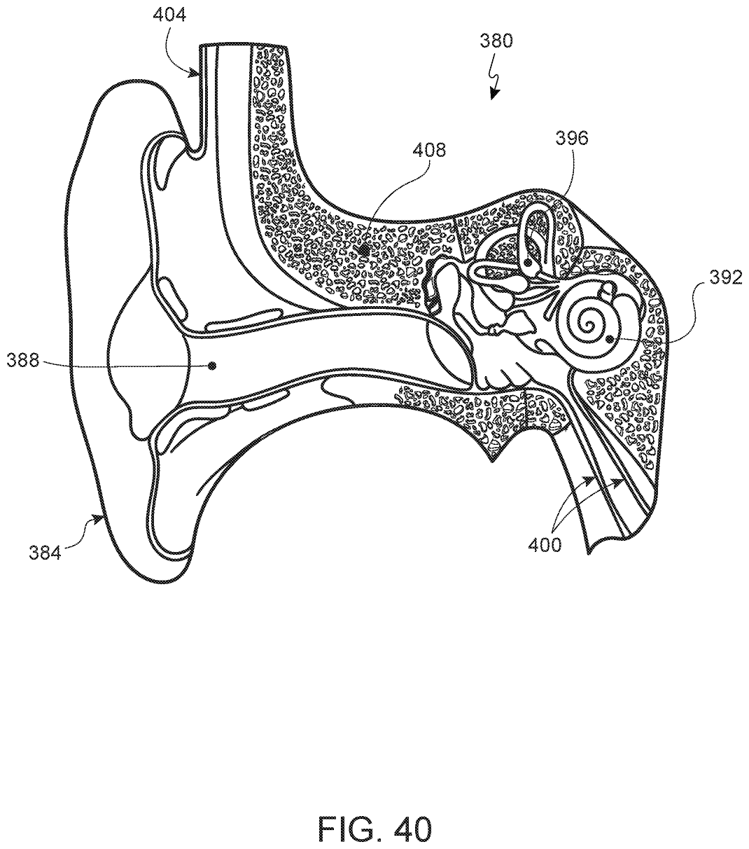

FIG. 40 illustrates the internal auditory system structures of a human head, generally 380, that are desirably effected by thermal therapy. Inner ear structures 380 include: outer ear 384; ear canal 388; cochlea 392; semi-circular canals (vestibular system) 396; nerve structures (auditory and vestibular 400; skin over the skull 404; and bone of the skull 408. It is believed that currently preferred thermal therapy devices act primarily to transfer heat from the structures of the inner ear by way of conduction through bone 408.

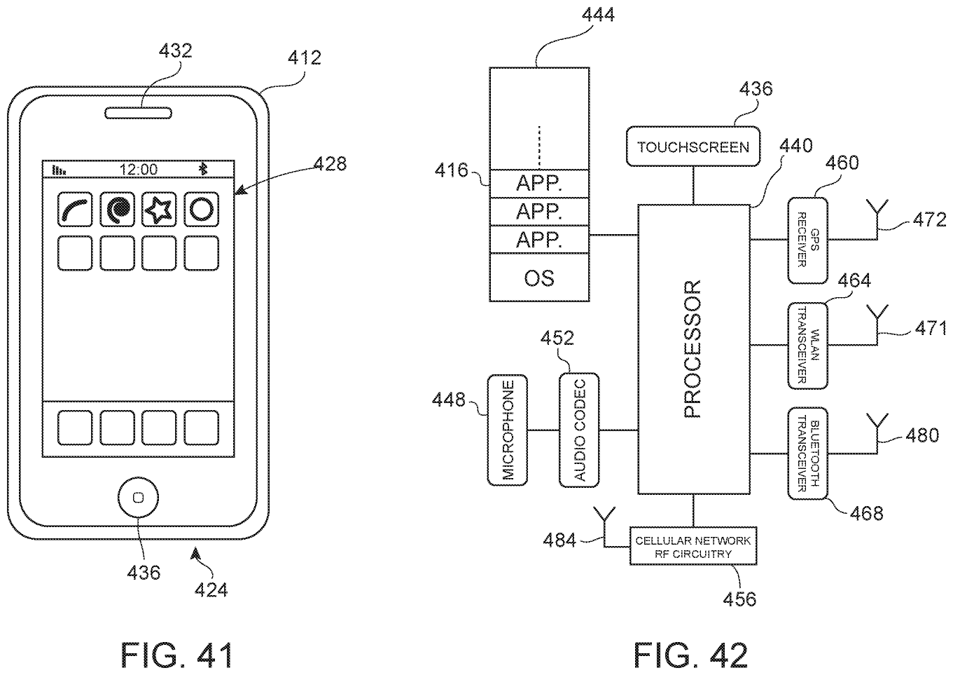

FIGS. 41-43 illustrate an exemplary mobile device platform+software package for interface with embodiments of cooling device(s). For this, a mobile phone 412, with an integrated software application 416, may be structured to communicate (wirelessly or wired) 420 with a cooling device. The communication is desirably 2-way: the cooling device sends/receives data from the mobile device, and the mobile device sends/receives data from the cooling device. Advantageously, a mobile phone includes: a microphone 424; a user interface 428 to convey information to a user; a speaker 432; and a user input control including button 436 to receive input from a user.

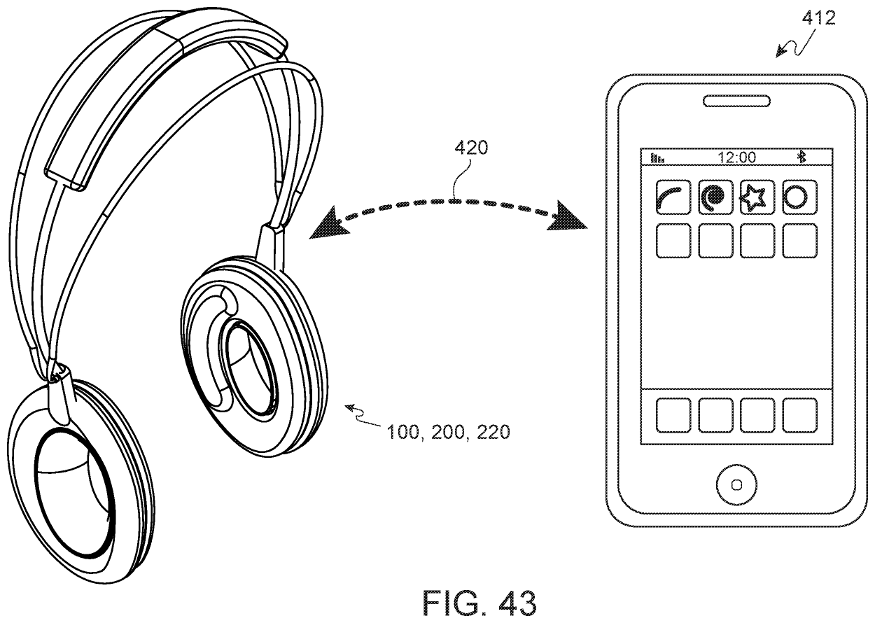

An exemplary mobile device platform generally indicated at 412 in FIG. 42 desirably includes: one or more software application(s) 416; a touch screen user input control 436; a microprocessor 440; on-board memory 444; a system microphone 448; an audio codec 452 to compress/decompress audio data; mobile device/cellular network communication circuitry 456; GPS transceiver module 460; wide/local area network (WLAN) transceiver module 464; Bluetooth transceiver 468; GPS antenna 472; WLAN antenna 476; Bluetooth antenna 480; and cellular antenna 484. As indicated generally in FIG. 43, a mobile platform may be placed in cooperation with a therapy device (e.g., passive or active) to monitor, detect, and provide therapy responsive to a perceived sonic event.

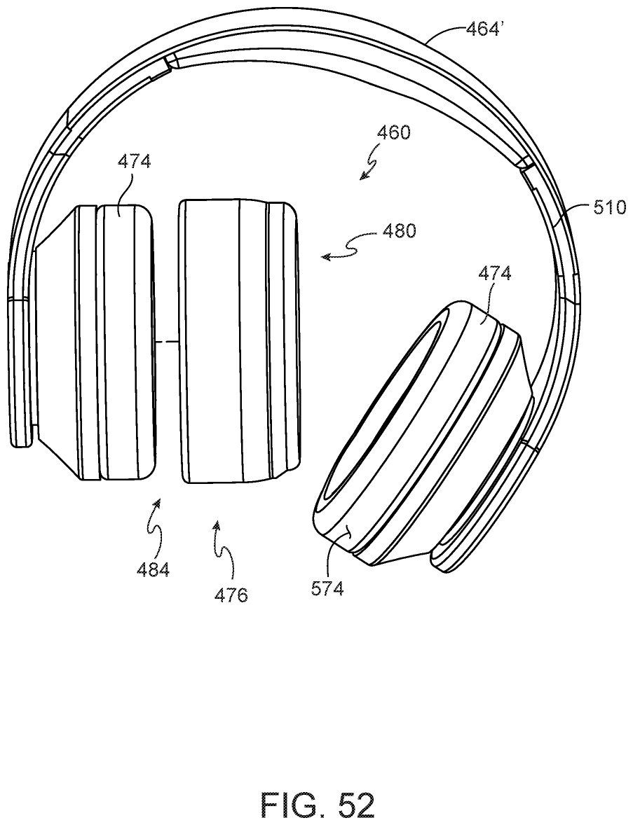

Reference will now be made to FIGS. 44 and 52. A workable device for use to apply thermal therapy to the head of a patient may be formed as a combination of one or more cooling ear cup accessory, generally 460, and a pair of commercially available auditory headphones 464 and 464′.

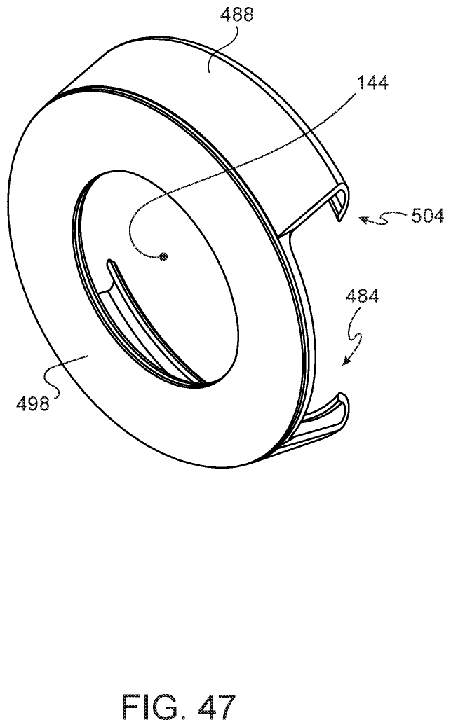

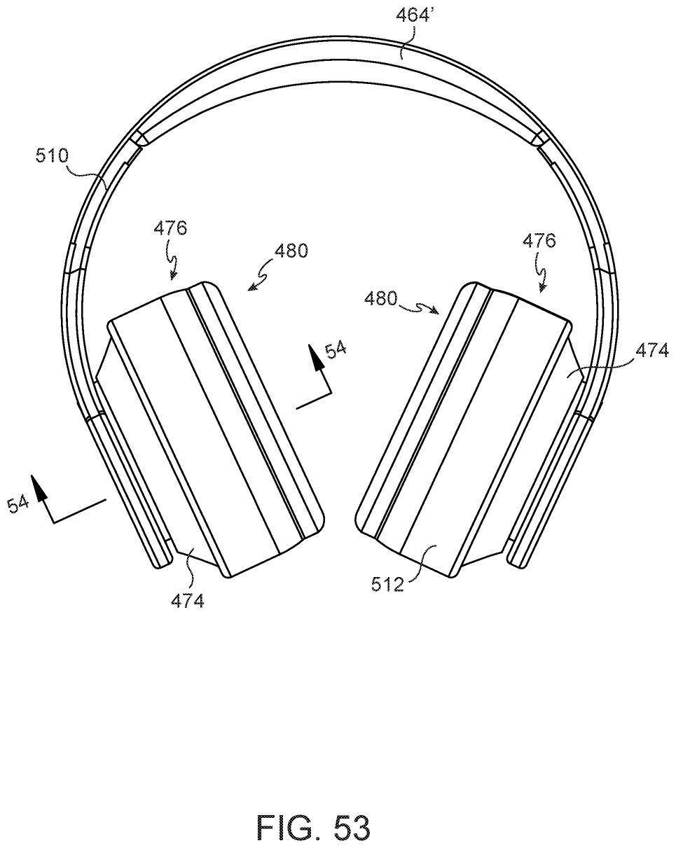

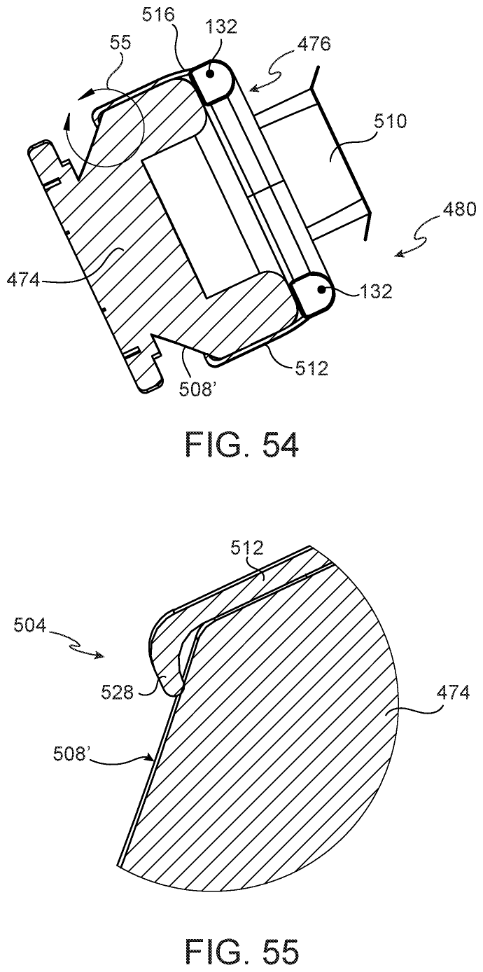

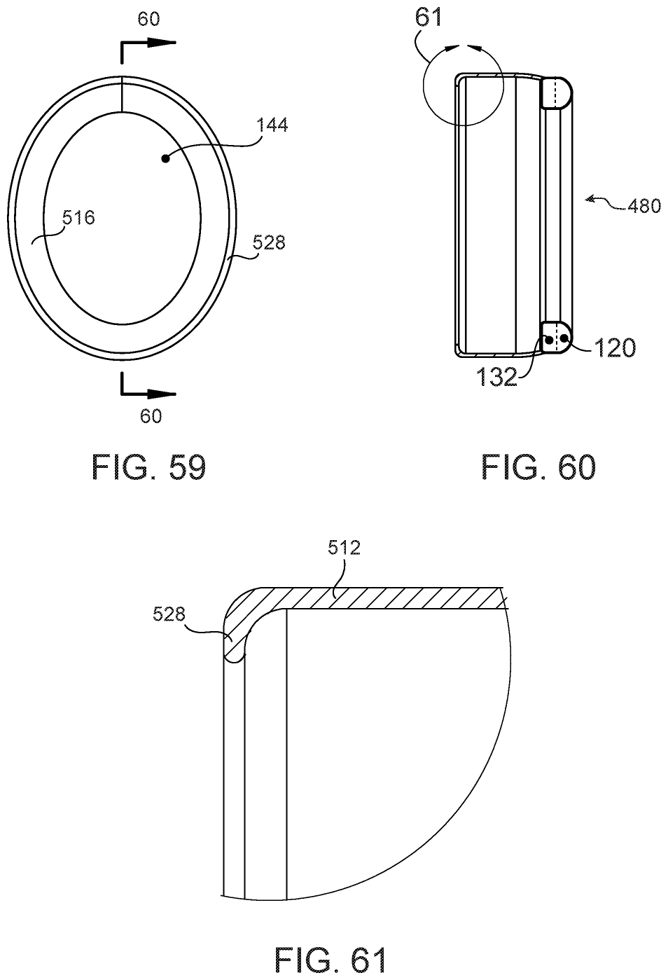

With reference to FIGS. 44 through 51, a first embodiment 472 of a cooling ear cup accessory 460 is structured to interface with a speaker-holding portion 474 (an earpiece) of auditory headphones 464 having a first generic shape. A second exemplary embodiment 476 of a cooling ear cup accessory 460 is illustrated in FIGS. 52 through 61. Cooling ear cup 476 is structured to interface with a speaker-holding portion 474 of auditory headphones 464′ having a second generic shape.

In either case, the illustrated cooling ear cups 472, 476 function to associate a cooling pack, generally 480, with a pair of headphones 464, 464′, so that the headphones assist in placing the cooling packs 480 in a desired position on a patient's head to apply thermal therapy onto the patient. An operable cooling pack 480 may be structured to include one or more element according to any of the devices mentioned above. It will be readily apparent that various structures may be arranged to associate a cooling pack 480 with headphones having any other configuration.

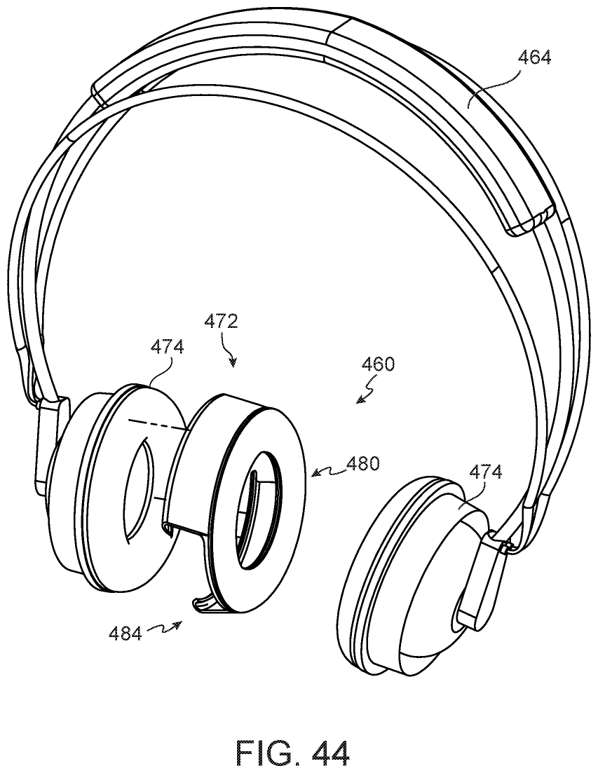

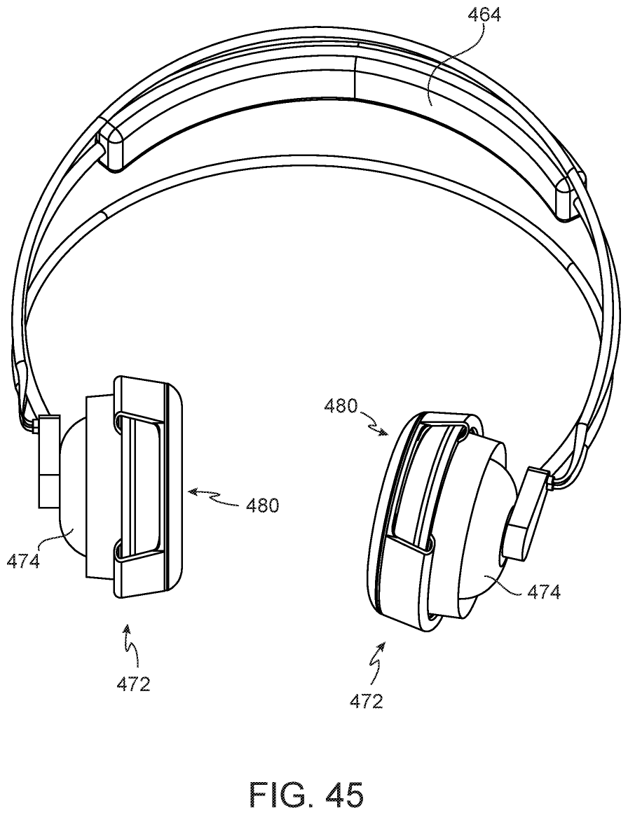

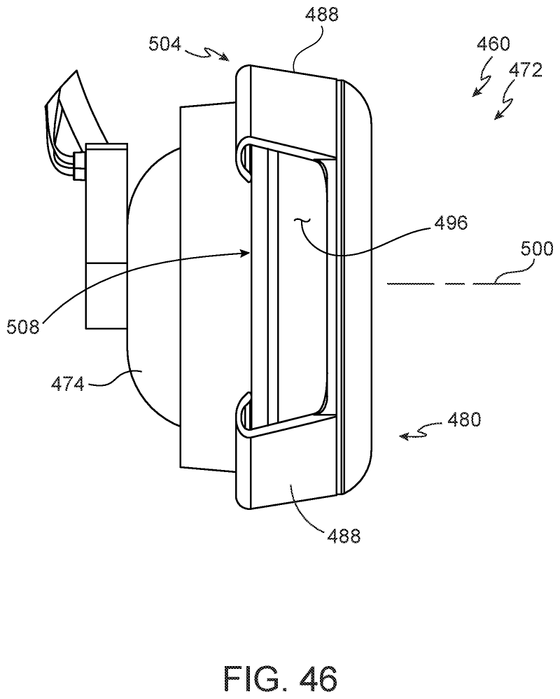

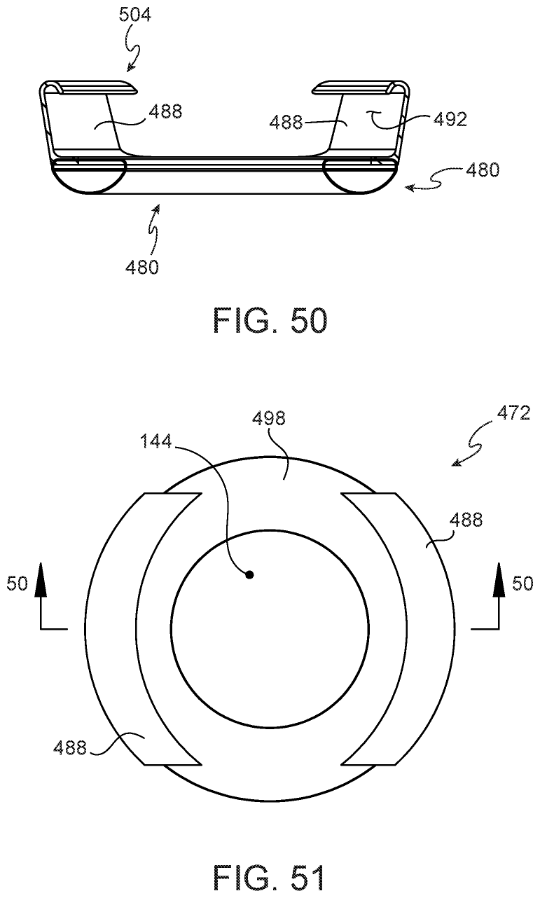

A cooling ear cup 472 is illustrated in a pre-installation position in FIG. 44, and a pair of cooling ear cups 472 are illustrated in their installed position in registration with earpieces 474 of headphones 464 in FIG. 45. Preferred embodiments of cooling cups typically include some sort of registration structure, generally indicated at 484, to enforce a degree of registered installation of a cup onto an earpiece 474. As illustrated in FIGS. 46 and 50, a workable registration structure 484 may include a pair of spaced apart upstanding arms 488 that are arcuate to form a somewhat cylindrical inside surface 492 against which a perimeter surface 496 of an earpiece 474 may be registered during installation of a cooling cup accessory 460 onto the earpiece. As pointed out in FIG. 51, arms 488 may be cantilevered from a base 498. An opposite side of base 498 may also support a cooling pack 480, or base 480 may be a portion of the cooling pack.

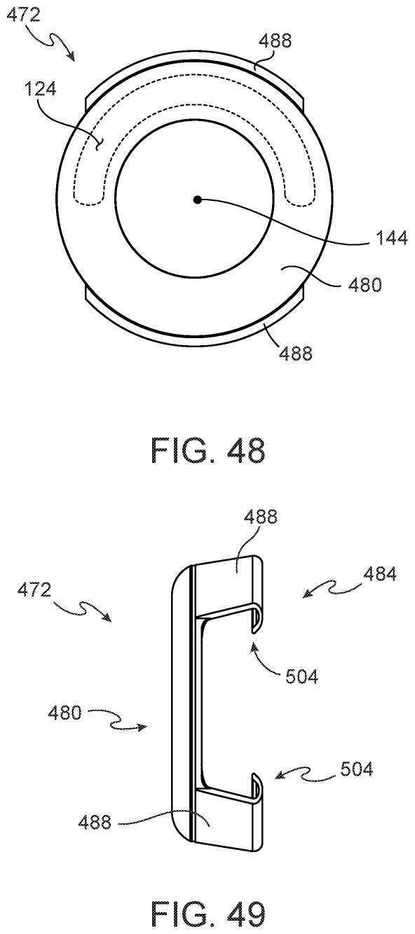

An installed cooling ear cup accessory 460 may sometimes be rotated about an adjustment axis 500 to a desired orientation (see FIG. 46). For example, an installed cooling ear cup may be rotated about adjustment axis 500 to dispose a heat transfer element 124 (see also FIG. 48) of the cooling cup 460 at a desired location for interface with an intended area for application of thermal therapy onto the head of a wearer. Sometimes, friction may be employed to resist movement of an installed cooling cup about adjustment axis 500. Other times, a portion of the registration structure 484 may interface with an earpiece 474 only in a pre-defined configuration.

Certain cooling ear cup accessories 460 may also include one or more element to resist unintended decoupling of a cooling cup from an earpiece 474 of a set of headphones. For example, in FIG. 46, arms 488 carry coupling elements, generally 504, structured to clip into engagement with cooperating surface 508 of earpiece 474 when the cooling ear cup accessory 460 is fully installed. A user may simply deflect the arms 488 to release the cup from the earpiece when desired. However, it is recognized that headphones typically have a self-bias that makes it optional to include structure to specifically resist decoupling. It is within contemplation that a user may even need to hold the cooling packs at a desired registered position during installation of a headphone/cooling pack assembly onto his/her head.

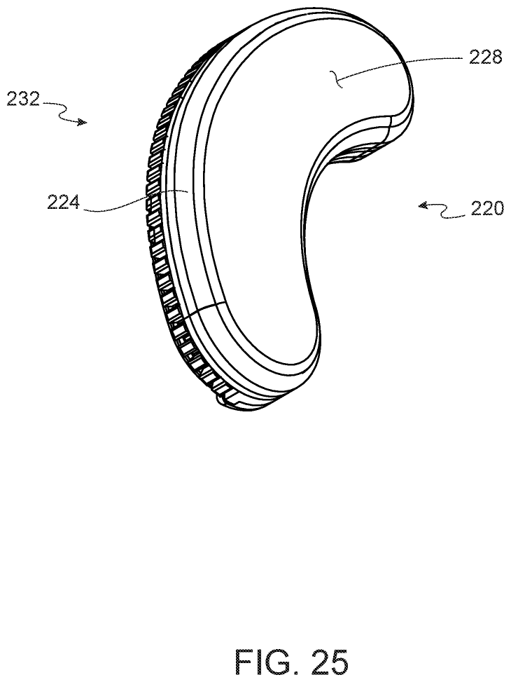

Certain details of construction for a cooling ear cup embodiment 476 will be discussed with reference now to FIGS. 52 through 61. It should be noted that FIGS. 24 and 25 are simplified and do not illustrate certain actual details, such as speakers and other internal structure of an earpiece 474. Earpieces 474 are carried conventionally by resilient band 510.

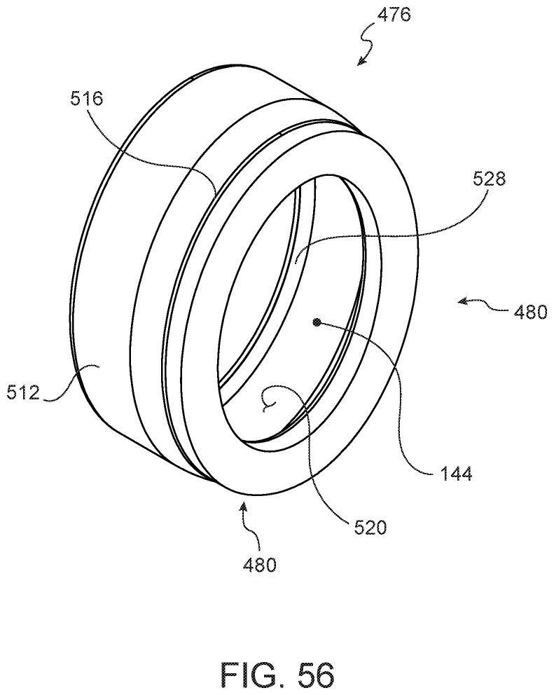

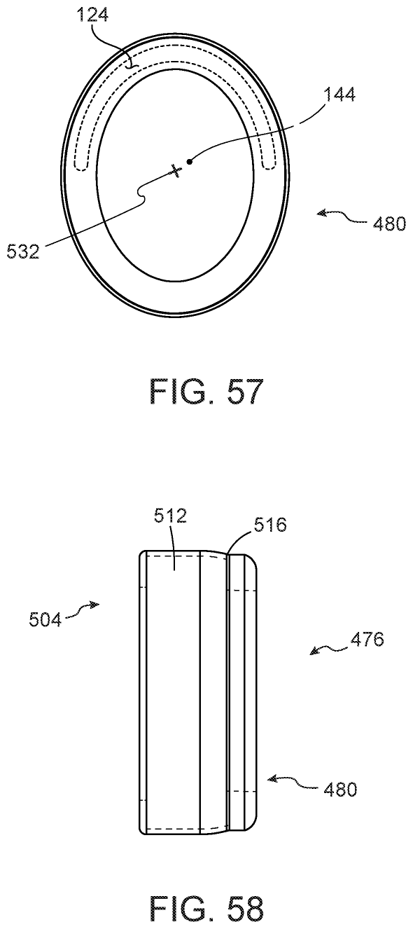

Cooling ear cup 476 includes a somewhat cylindrical upstanding wall 512 that is attached at a proximal end to a base 516. A cooling pack 480 may conveniently be affixed to, or otherwise carried on, an opposite side of base 516. Wall 512 defines registration structure 484, including an inner surface 520, which may engage with a perimeter surface 524 of earpiece 474 to register a cooling ear cup 476 in installed engagement with earpiece 474. As illustrated in FIG. 57, wall 512 may be configured to define a somewhat oval cross-section shape. Such a noncircular shape may be of use to define a preferred orientation for an installed cooling ear pack. For example, the installed orientation may dispose a heat transfer element in a desired localized interface area with respect to a head and ear of a therapy patient. The exemplary noncircular cross-section resists departure of the cooling ear pack from a desired orientation about installation axis 532 with respect to an ear of a wearer of the headphones.

The distal free end of wall 512 may optionally carry coupling structure, generally indicated at 504. A workable coupling structure 504 is configured to resist undesired separation of a cooling cup from an earpiece. Illustrated coupling structure 504 includes a flange 528 that is configured to form a structural interference with cooperating surface 508′ of an earpiece 474 when the cooling ear cup 476 is fully installed on the ear piece 474. A workable wall 512 may be made from a resilient and elastic material to stretch or deflect during installation and removal, and to strive to return to an original configuration absent an imposed biasing force to the contrary. Although wall 512 is illustrated as being uninterrupted around a perimeter, it is within contemplation to provide one or more axially oriented slot, break, or gap, to create a plurality of fingers having equivalent coupling functionality.

As illustrated in FIG. 54, a cooling pack 480 may include a bulk cavity 132 to hold heat transfer media. In certain embodiments, a portion of the bulk cavity 132 may be disposed to contact the head of a patient, and also function as a heat transfer cavity 120. As illustrated in FIG. 60, a bulk cavity 132 may be in thermal communication with a dedicated heat transfer cavity 120 that also holds a heat transfer media. The heat transfer media(s) in each cavity may be selected to deliver a desired thermal therapy. A heat transfer cavity 120 may be disposed to contact a wearer's head in a contact patch or surface 124 that completely circumscribes the ear canal. However, it is currently preferred to provide a heat transfer cavity 120 with a contact surface 124 that circumscribes only a portion of the entire perimeter of the ear canal (see FIG. 57). A bulk cavity 132 may still be configured to circumscribe the ear canal while its associated heat transfer cavity circumscribes only a portion of that distance.

Embodiments structured according to certain aspects of this invention may include a thermochromic ink to indicate a current temperature of the device to the user. Thermochromic ink is a chemical compound that changes color based on temperature. For example, a graphic printed using this ink, located on visible portion of a heat transfer element may appear “blue” in color (or some other color) when the bladder is at 5 degrees C. When the device increases in temperature to say, 20 degrees C., the “blue” color of the graphic may change color to white (or some other color). This color change indicates to the user that the device is either ready, or not ready, for use. The ink may be printed on a heat transfer element, or some other visible surface of the device. The ink may also be contained within the heat transfer media or fluid within a cavity, or it may be contained within the material used to create a housing, or a media cavity or bladder itself. Thermochromic ink may be applied to any surface, or disposed to influence the visible appearance of any surface, that is effective to inform a user of the operating temperature status of a thermal therapy device.

While aspects of the invention have been described in particular with reference to certain illustrated embodiments, such is not intended to limit the scope of the invention. The present invention may be embodied in other specific forms without departing from its spirit or essential characteristics. For one example, one or more element may be extracted from one described or illustrated embodiment and used separately or in combination with one or more element extracted from one or more other described or illustrated embodiment(s), or in combination with other known structure. The described embodiments are to be considered as illustrative and not restrictive. Obvious changes within the capability of one of ordinary skill are encompassed within the present invention.

The scope of the invention for which a monopoly position is currently desired is indicated by the appended claims rather than by the foregoing description. All changes which come within the meaning and range of equivalency of the claims are to be embraced within their scope.

What is claimed is:

1. An apparatus, comprising:
a cooling pack comprising a heat transfer element; and
registration structure associated with the cooling pack, the registration structure being configured to removably interface in installed registration with an earpiece of a pair of commercially available auditory headphones to dispose the heat transfer element in contact with a localized area of the head of a wearer of the headphones.

2. The apparatus according to claim 1, wherein:
the heat transfer element is configured such that the localized area extends only partially around a circumference of an ear of the wearer.

3. The apparatus according to claim 2, wherein:
the cooling pack is structured and arranged in harmony with the registration structure to maintain the heat transfer element at a posterior position with respect to the ear canal of the ear.

4. The apparatus according to claim 1, wherein:
the registration structure comprises a coupling element configured to resist undesired decoupling of the cooling pack from the earpiece.

5. The apparatus according to claim 4, wherein:
the coupling element is configured to form an interference fit against a cooperating portion of the headphones.

6. The apparatus according to claim 1, wherein:
the registration structure is configured to form an interference fit against a cooperating portion of the headphones.

7. The apparatus according to claim 4, wherein:
the coupling element is configured to form a friction fit against a cooperating portion of the headphones.

8. The apparatus according to claim 2, wherein:
the cooling pack comprises a bulk cavity carrying thermal media disposed in thermal communication with the heat transfer element; and
the bulk cavity extends in a circumscribing direction entirely around the ear.

9. The apparatus according to claim 8, wherein:
thermochromic ink is disposed in association with the apparatus to inform of the operational status of the apparatus.

10. The apparatus according to claim 1, wherein:
the cooling pack is passive.

11. The apparatus according to claim 1, wherein:
the cooling pack comprises an electrically active thermal element.

12. The apparatus according to claim 1, wherein:
the heat transfer element comprises a floor of a contact cavity; and
a first thermal media is disposed inside the contact cavity.

13. The apparatus according to claim 12, wherein:
the floor is transversely flexible and conformable to accommodate and conform under compression against the skin in the vicinity of the ear.

14. The apparatus according to claim 12, further comprising:
a bulk cavity disposed in thermal communication with the contact cavity; and
a thermal media is disposed inside the bulk cavity.

15. The apparatus according to claim 14, wherein:
the bulk cavity is further disposed in fluid communication with the contact cavity.

16. The apparatus according to claim 14, wherein:
the bulk cavity is sized to hold between two-times and about twenty five-times the volume of a heat transfer media that is held in the contact cavity.

17. The apparatus according to claim 14, wherein:
a boundary between the contact cavity and the bulk cavity is defined by a step-change in cross-section at the boundary, heat transfer between the contact cavity and bulk cavity occurring across a cross-section disposed at the step-change location.

18. The apparatus according to claim 14, wherein:
the bulk cavity is insulated to resist direct heat transfer into the bulk cavity from the local environment.

19. The apparatus according to claim 14, wherein:
the bulk cavity is insulated to resist direct heat transfer into the bulk cavity from the head and/or structured to avoid contact with the head.

20. The apparatus according to claim 14, wherein:
the bulk cavity is insulated to resist direct heat transfer into the bulk cavity from the ear.
